(19) United States
(12) Patent Application Publication
(10) Pub. No.: US 2024/0425866 A1
(43) Pub. Date: Dec. 26, 2024

(54) PARN AS A BIOMARKER AND THERAPEUTIC TARGET

(71) Applicant: The Regents of the University of Colorado, a body corporate, Denver, CO (US)

(72) Inventors: Roy R. Parker, Boulder, CO (US); Shukla Siddharth, Boulder, CO (US)

(21) Appl. No.: 18/794,630

(22) Filed: Sep. 12, 2024

Related U.S. Application Data
(63) Continuation of application No. 17/266,502, filed on Feb. 5, 2021, now abandoned.
(60) Provisional application No. 62/715,688, filed on Aug. 7, 2018.

(51) Int. Cl. C12N 15/113; A61K 31/704; A61K 31/7125; A61P 35/00; C12Q 1/6886

(52) U.S. Cl. CPC C12N 15/1137; A61K 31/704; A61K 31/7125; A61P 35/00; C12Q 1/6886; C12N 2310/11; C12N 2310/315; C12N 2320/31; C12Q 2600/158

(57) ABSTRACT

Increased PARN as an indicator of a cancer involving loss or reduction in p53 function. PARN is also provided as a therapeutic target for treating a cancer involving loss or reduction in p53 function. Methods of treating a subject having a cancer involving loss or reduction in p53 function based on the level of PARN in a test sample obtained from the subject and administering an effective amount of a PARN inhibitor, alone or in conjunction with another chemotherapeutic agent, to the subject to treat the cancer.

### CROSS REFERENCE TO RELATED APPLICATION

This application is a continuation of U.S. application Ser. No. 17/266,502, filed Feb. 5, 2021, which is claimed the benefit of PCT Application No. PCT/US2019/045540 having an international filing date of Aug. 7, 2019, which designated the United States, which PCT application claimed the benefit of U.S. Application No. 62/715,688, filed Aug. 7, 2018, all of which are incorporated by reference in their entirety.

### GOVERNMENT INTEREST

This invention was made with government support under grant number GM045443, awarded by National Institutes of Health. The United States government has certain rights in the invention.

### SEQUENCE LISTING

The instant application contains contents of the electronic sequence listing (90245.00313-Sequence-Listing.xml; Size: 20,542 bytes; and Date of Creation: Sep. 12, 2024) is herein incorporated by reference in its entirety.

### TECHNICAL FIELD

The invention relates to medical uses and methods for modulating gene expression and treating cancer using poly (A)-specific ribonuclease (PARN) inhibitors, and to methods and uses for overcoming resistance of cancer cells to chemotherapy, including selecting PARN inhibitors for use in treating cancer in a subject, both in the initial selection of PARN inhibitors and for addressing the development of acquired drug resistance that occur in the course of treatment.

### BACKGROUND OF INVENTION

The adenylation of 3′ ends of cellular RNAs by poly (A) polymerases plays a critical role in the function and stability of both mRNAs and non-coding RNAs. PARN is a processive mammalian poly (A)-specific ribonuclease that has previously been shown to remove poly (A) tails from the 3′ ends of mRNAs. Recent work has shown that PARN also regulates the stability of several ncRNAs in mammalian cells, including scaRNAs, human telomerase RNA (hTR), piRNAs and Y RNAs, suggesting that the deadenylation activity of PARN is important for regulating the stability of a variety of RNAs in mammalian cells.

miRNAs are small 21-23 nt non-coding RNAs that regulate gene expression in eukaryotic cells through base pairing with their target mRNAs. miRNAs are transcribed as long primary transcripts (pri-miRNA), which are trimmed by the endonuclease Drosha to generate the precursor miRNA (pre-miRNA) containing the miRNA stem-loop. The pre-miRNA is subsequently cleaved by Dicer to generate the mature miRNA, which assembles with Argonaute and GW182 along with other proteins to form the RNA-induced silencing complex (RISC). While the role of miRNAs in regulating gene expression is well studied, the mechanism(s) that globally regulates the stability of miRNAs in mammalian cells are not fully understood. Previous work has suggested that XRN2-mediated 5′ to 3′ degradation can regulate the stability of some miRNAs in model organisms. More recent work has shown that Tudor S/N mediated endonucleolytic cleavage can also regulate the stability of some miRNAs in mammalian cells.

miRNAs are known to be modified by non-templated U or A additions at the 3′ end in diverse cell types and organisms. In plants, Henl-mediated 3′ end methylation of the 2′-OH moiety has been shown to protect endogenous plant siRNAs and miRNAs from uridylation and degradation by SND1. In black cottonwood plant, adenylation of the 3′ end is a feature of miRNA degradation products, and adenylation can also reduce the degradation of plant miRNAs. In the alga Chlamydomonas, Mut68 uridylates the 3′ ends of endogenous siRNAs and miRNAs, suggesting a conserved function of 3′ end modification of small RNAs in different organisms.

The best studied example of 3′ end non-templated addition in mammalian miRNAs is the uridylation of the let-7 pre-miRNA by non-canonical uridylases TUT4/TUT7. Tut4/Tut7 are recruited by the RNA binding protein LIN28 to the pre-let-7, which leads to polyuridylation of the pre-let-7 3′ end and affects its processing into mature let-7, thereby playing a role in regulating let-7 miRNA levels and function in animal development. It has also been proposed that monouridylation of some let-7 pre-miRNAs as opposed to polyuridylation is important for their processing to mature let-7 miRNA in HeLa cells, suggesting that the activity of Tut4/Tut7 may be regulated in mammalian cells to maintain a balance between let-7 processing and degradation. Uridylation of pre-let-7 leads to the recruitment of the 3′ to 5′ exonuclease DIS3L2, and DIS3L2 degrades polyuridylated pre-let-7 in undifferentiated stem cells. Uridylation of pre-miRNAs and miRNAs has also been shown to occur on other families of miRNAs in diverse cell types, suggesting that uridylation of pre-miRNAs and mature miRNAs is a general feature of miRNA regulation.

Adenylation at the 3′ end has also been shown to occur for some miRNAs, although it is suggested to be less frequent compared to uridylation. The best understood example of miRNA adenylation is GLD2-mediated monoadenylation of miR-122, which enhances the stability and function of miR-122 in mammalian cells. Similarly, monoadenylation of the 3′ end by GLD2 also enhanced the stability of some other miRNAs in human fibroblasts. In contrast, PAPD5-mediated adenylation has been proposed to destabilize miR-21 in human cancer cell lines. In Drosophila, wisp-mediated adenylation of the 3′ end destabilizes maternal miRNAs in eggs and is an important step in maternal miRNA clearance. For monoadenylated miR-122 in liver cells, PARN has been proposed to be the enzyme responsible for the degradation of miR-122 when it is adenylated by GLD2. However, the mechanisms that regulate miRNA 3′ end adenylation and deadenylation, and the role of PARN in this process, are not well understood.

Dyskeratosis Congenita (DC) is caused by genetic defects in components of the telomerase holoenzyme in human cells and leads to bone marrow failure and cancer. While most mutations associated with DC pathogenesis are in genes important for human telomerase RNA (hTR) assembly (DKC1) or telomerase RNA stability (TERC), mutations in PARN were shown to cause a severe form of DC known as Hoyeraal-Hreidarsson syndrome, which causes abnormally short telomeres and congenital. Subsequently, it was shown that loss of PARN leads to defective 3′ end maturation of hTR, leading to oligoadenylation by PAPD5 and 3′ to 5′ degradation by EXOSC10 in the nucleus, or cytoplasmic export and decapping and 5′ to 3′ degradation by DCP2/XRN1. While loss of telomerase RNA function explains telomere shortening in DC patients, it doesn't explain the pleiotropic and severe phenotype of the disease caused by PARN mutations.

In view of the need for further cancer therapies, this disclosure provides methods of treating and diagnosing cancer related to PARN expression and activity that effects oncogene expression.

### SUMMARY OF INVENTION

The present inventors hypothesized that PARN deficiency affects the stability of miRNAs in human cells, which could explain the severe phenotype of PARN deficiency in DC patients. They have now surprisingly shown that in Hela cells, PARN affects the levels of several miRNAs both positively and negatively. Further, they have shown that PARN protects miRNAs from degradation by removing adenosines from their 3′ ends that are added by the poly (A) polymerase PAPD5. In the absence of PARN, 3′ end adenylation leads to recruitment of the cytoplasmic exonucleases DIS3L or DIS3L2, which leads to miRNA degradation. They have also found that several miRNAs that are decreased in PARN depleted cells target the p53 mRNA, and that PARN knockdown leads to a very strong upregulation of p53 protein levels in Hela cells, with or without DNA damage. They have also shown that PARN knockdown sensitizes Hela cells to chemotherapeutic agents, leading to cell cycle arrest and apoptosis. These findings explain why PARN mutations lead to a severe phenotype of DC in patients, because chronic upregulation of p53 signaling could negatively affect cell growth and development in these patients at a very young age. Additionally, the use of PARN inhibitors combined with chemotherapy could be a therapeutic strategy to treat a subset of cancers that are caused by repressed wild-type p53 protein.

Thus, this disclosure provides methods of reducing the severity of one or more symptom(s) of cancer in a patient, and/or identifying a cancer patient that may selectively benefit from the administration of one or more PARN inhibitor(s) or the administration of the combination of one or more PARN inhibitor(s) and one or more chemotherapeutic agent(s), and/or diagnosing a chemotherapy-resistant or chemotherapy-sensitive cancer by measuring one or more feature(s) in cancer cell(s) from a patient selected from levels of poly (A)-specific ribonuclease (PARN), levels of phosphorylated PARN, and determining from the measurements whether the cancer cell(s) in the patient has one or more feature(s) of an activated PARN and/or an inactivated p53 signaling pathway relative to these features in a control sample; and administering to a patient determined to have a cancer cell having one or more the feature(s) of an activated PARN and/or an activated p53 signaling pathway one or more P53 inhibitor(s) for a time and in an amount sufficient to reduce the severity of one or more symptom(s) of cancer in the patient.

Additional embodiments of the above methods include measuring one or more feature(s) in the cancer cell(s) selected from tumor protein-53 (p53) mRNA or protein levels, and cyclin-dependent kinase inhibitor 1 (p21) expression or activity, and determining from these measurements whether the cancer cell(s) in the patient have one or more feature(s) of an inactivated p53 signaling pathway selected from decreased p53 mRNA or protein levels, expression of a mutant or truncated p53 with decreased expression or activity, and decreased p21 expression or activity, relative to these features in a control sample. These methods may further include administering to a patient determined to have a cancer cell having one or more the feature(s) of an inactivated p53 signaling pathway a PARN inhibitor for a time and in an amount sufficient to reduce the severity of one or more symptom(s) of cancer in the patient. Further embodiments of any of the above methods further comprise the step of administering one or more chemotherapeutic agent(s) (e.g., a chemotherapeutic agent that induces DNA damage) to the patient.

In any of the above methods, the control sample in step (ii) may be a non-cancerous cell or a cell untreated with a genotoxic agent and/or the control sample in step (v) is a non-cancerous cell.

In any of the above methods, the PARN inhibitor may be a small molecule, or an siRNA molecule or a nucleobase oligomer containing a sequence complementary to at least 10 consecutive nucleotides of a nucleic acid sequence encoding a PARN protein, or a peptide that may be covalently-linked to a moiety capable of tranlocating across a biological membrane (e.g., a moiety that contains a penetratin peptide or a TAT peptide).

In any of the above methods, the patient may have previously received at least one dosage of a chemotherapeutic agent. In additional embodiments of the above methods, the control sample is a cancer cell or non-cancerous cell treated with a genotoxic agent and/or the control sample is a non-cancerous cell.

This disclosure further provides methods of treating a cancer patient diagnosed as having a chemotherapy-resistant cancer by any of the above methods, requiring the step of administering to the patient one or more PARN inhibitor(s). These methods may further include administering one or more chemotherapeutic agent(s) (e.g., a chemotherapeutic agent that induces DNA damage) to the patient.

In any of the above methods, the chemotherapeutic agent may be selected from the group of: alemtuzumab, altretamine, aminoglutethimide, amsacrine, anastrozole, azacitidine, bleomycin, bicalutamide, busulfan, capecitabine, carboplatin, carmustine, celecoxib, chlorambucil, 2-chlorodeoxyadenosine, cisplatin, colchicine, cyclophosphamide, cytarabine, cytoxan, dacarbazine, dactinomycin, daunorubicin, docetaxel, doxorubicin, epirubicin, estramustine phosphate, etodolac, etoposide, exemestane, floxuridine, fludarabine, 5-fluorouracil, flutamide, formestane, gemcitabine, gentuzumab, goserelin, hexamethylmelamine, hydroxyurea, hypericin, ifosfamide, imatinib, interferon, irinotecan, letrozole, leuporelin, lomustine, mechlorethamine, melphalen, mercaptopurine, 6-mercaptopurine, methotrexate, mitomycin, mitotane, mitoxantrone, nilutamide, nocodazole, paclitaxel, pentostatin, procarbazine, raltitrexed, rituximab, rofecoxib, streptozocin, tamoxifen, temozolomide, teniposide, 6-thioguanine, topotecan, toremofine, trastuzumab, vinblastine, vincristine, vindesine, and vinorelbine.

In any of these methods, the cancer may be selected from acoustic neuroma, acute leukemia, acute lymphocytic leukemia, acute monocytic leukemia, acute myeloblastic leukemia, acute myelocytic leukemia, acute myelomonocytic leukemia, acute promyelocytic leukemia, acute erythroleukemia, adenocarcinoma, angiosarcoma, astrocytoma, basal cell carcinoma, bile duct carcinoma, bladder carcinoma, brain cancer, breast cancer, bronchogenic carcinoma, cervical cancer, chondrosarcoma, chordoma, choriocarcinoma, chronic leukemia, chronic lymphocytic leukemia, chronic myelocytic leukemia, colon cancer, colon carcinoma, craniopharyngioma, cystadenocarcinoma, embryonal carcinoma, endotheliosarcoma, ependymoma, epithelial carcinoma, Ewing's tumor, glioma, heavy chain disease, hemangioblastoma, hepatoma, Hodgkin's disease, large cell carcinoma, leiomyosarcoma, liposarcoma, lung cancer, lung carcinoma, lymphangioendotheliosarcoma, lymphangiosarcoma, macroglobulinemia, medullary carcinoma, medulloblastoma, melanoma, meningioma, mesothelioma, myxosarcoma, neuroblastoma, non-Hodgkin's disease, oligodendroglioma, osteogenic sarcoma, ovarian cancer, pancreatic cancer, papillary adenocarcinomas, papillary carcinoma, pincaloma, polycythemia vera, prostate cancer, rhabdomyosarcoma, renal cell carcinoma, retinoblastoma, schwannoma, sebaceous gland carcinoma, seminoma, small cell lung carcinoma, squamous cell carcinoma, sweat gland carcinoma, synovioma, testicular cancer, uterine cancer, Waldenstrom's fibrosarcoma, and Wilm's tumor. In additional examples of any of the above methods, the cancer cell(s) are from a biopsy sample from the patient.

The invention further provides kits for diagnosing a chemotherapy-resistant or chemotherapy-sensitive cancer in a patient.

This Summary of the Invention is neither intended nor should it be construed as being representative of the full extent and scope of the present invention. Moreover, references made herein to “the present invention,” or aspects thereof, should be understood to mean certain embodiments of the present invention and should not necessarily be construed as limiting all embodiments to a particular description. The present invention is set forth in various levels of detail in the Summary of the Invention as well as in the attached drawings and the Description of Embodiments and no limitation as to the scope of the present invention is intended by either the inclusion or non-inclusion of elements, components, etc. in this Summary of the Invention. Additional aspects of the present invention will become more readily apparent from the Description of Embodiments, particularly when taken together with the drawings.

### BRIEF DESCRIPTION OF FIGURES

FIGS. 1A-1C demonstrate that PARN depletion affects several miRNAs and affects the stability of both precursor and mature miRNAs. FIG. 1A is a line plot depicting changes in miRNA levels in PARN knockdown cells compared to control for four biological replicates. Each dot represents an individual miRNA. Red dots and green intercepts depict lower and upper cutoffs for differentially expressed miRNAs (<0.7× or >1.5X in PARN KD compared to control). FIG. 1B shows representative northern blots of miR-1, miR-380-5p, miR-181b-5p and miR-21-5p in control and PARN knockdown cells (Average+/−S.D. for five biological replicates for northern blots). FIG. 1C shows the relative miR-1 and miR-380 pre- and mature miRNA levels in control and PARN knockdown cells quantified using qRT-PCR and normalized to 5s rRNA (Average+/−S.D. for three biological replicates).

FIGS. 2A-2H demonstrate that PAPD5-mediated 3′ end adenylation destabilizes miRNAs and leads to their degradation by DIS3L or DIS3L2. FIG. 2A shows representative northern blots for mature miRNAs PARN and PAPD5 co-knockdown cells. Histogram represents relative miRNA levels for the indicated miRNA species (Average+/−S.D. for five biological replicates). FIG. 2B shows representative northern blots for different miRNAs in PAPD5 knockdown cells (Average+/−S.D. for four biological replicates). FIG. 2C shows representative northern blots for different miRNAs in DIS3L and DIS3L2 knockdown cells (Average+/−S.D. for four biological replicates). FIGS. 2D and 2E are histograms depicting a proportion of oligoadenylated species for miR-21-5p and miR-181b-5p, respectively, in PARN knockdown and PARN and PAPD5 co-knockdown cells. Z scores were calculated using two proportion Z-test, and converted to P values. (*** P<0.001). FIG. 2F is a histogram depicting a proportion of oligoadenylated species for miR-21-5p and miR-181b-5p, respectively, in control knockdown and DIS3L2 knockdown cells. Z scores were calculated using two proportion Z-test, and converted to P values. (*** P<0.001). FIG. 2G is a histogram depicting proportion of oligouridylated species for miR-181b-5p in control and DIS3L2 knockdown cells. FIG. 2H is a histogram depicting proportion of oligoadenylated species for miR-181b-5p in control and DIS3L knockdown cells. Z scores were calculated using two proportion Z-test, and converted to P values. (*** P<0.001).

FIGS. 3A-3E demonstrate that PARN regulates a miRNA circuit that controls p53 levels in human cells. FIG. 3A depicts miRNAs that affect p53 signaling pathway are affected in PARN knockdown cells. miRNAs in red are downregulated in PARN knockdown cells, while miRNAs in green are upregulated in PARN knockdown cells. FIG. 3B shows representative western blots depicting p53 protein levels in PARN knockdown cells under various conditions. FIG. 3C shows representative western blots depicting p53 and PARN protein levels in PARN knockdown and rescued cells. FIG. 3D shows p53 mRNAs levels as measured using RT-qPCR in PARN knockdown cells. (Average+/−S.D. for three biological replicates). FIG. 3E shows representative western blots depicting p53 protein levels in 293T wild type and Dicer knockout cells. (Average +/−S.D. for three biological replicates).

FIGS. 4A-4E demonstrate that PARN knockdown increases sensitivity of Hela cells to chemotherapeutic agents. FIG. 4A shows representative western blots depicting p53 and PARN protein levels in various knockdown conditions. FIG. 4B shows the quantification of p53 protein levels in various conditions using western blotting. Histogram represents five biological replicates for untreated and four biological replicates for Doxorubicin-treated cells (Average+/−S.D.). FIG. 4C shows representative bright field images of control and PARN knockdown Hela cells at indicated time points after Doxorubicin treatment. (Scale bar: 300 μm). FIGS. 4D and 4E show viable cells under various knockdown and treatment conditions as measured using trypan blue staining assay (Average+/−S.D for three biological replicates).

FIG. 5 depicts a model for the regulation of miRNA stability by 3′ end adenylation and deadenylation by PARN and PAPD5, and degradation by DIS3L or DIS3L2. miRNAs are transcribed as primary transcripts in the nucleus and processed to pre-miRNAs by the Drosha endonuclease, which are exported to the cytoplasm and matured by Dicer. The pre-miRNA could be subjected to adenylation and deadenylation at the 3′ end by the competing activities of PARN and PAPD5 in the cytoplasm leading to degradation by DIS3L. Pre-miRNA is also uridylated and degraded by the activities of TUT/DIS3L2. Mature miRNA could be targeted to adenylation by PAPD5 either through release from RISC or due to binding to complementary target mRNAs. PARN limits the adenylation of mature miRNA strands, and in PARN deficient cells, DIS3L or DIS3L2 can degrade specific miRNAs.

FIG. 6 is a line plot depicting changes in miRNA levels in DIS3L knockdown cells compared to control for two biological replicates, showing that DIS3L regulates the levels of multiple miRNAs in human cells. Each dot represents an individual miRNA. Red and green intercepts depict lower and upper cutoffs for differentially expressed miRNAs (<0.7x or >1.5X in DIS3L KD compared to control).

FIG. 7 is a line plot depicting changes in miRNA levels in DIS3L2 knockdown cells compared to control for two biological replicates, showing that DIS3L2 regulates the levels of multiple miRNAs in human cells. Each dot represents an individual miRNA. Red and green intercepts depict lower and upper cutoffs for differentially expressed miRNAs (<0.7x or >1.5X in DIS3L2 KD compared to control).

FIG. 8 is a model for competing activities of PARN and DIS3L2 on 3′ oligo (A) tails of miRNAs. 3′ end oligoadenylation of miRNAs is regulated by the deadenylation activity of PARN. During this reversible process, DIS3L2 outcompetes PARN and commits a miRNA for degradation by recognizing the 3′ end oligo (A) tails, and eventually degrades the miRNA. When DIS3L2 is knocked down, PARN can deadenylate the miRNA 3′ end, which leads to shorter oligo (A) tails on miRNAs.

FIG. 9 is a bar graph depicting percentage of propidium iodide stained Hela cells in various phases of the cell cycle showing that p53 induction in PARN knockdown Hela cells leads to cell cycle abnormalities compared to control cells. (Average+/−S.D. for three biological replicates). * P<0.05, ** P<0.01, two-tailed unpaired Student's T-test.

FIG. 10 shows representative western blots for HPV E6 protein levels in Hela cells upon indicated treatments, showing that PARN knockdown does not affect the levels of the HPV E6 oncoprotein in Hela cells. Gapdh was used a loading control.

FIGS. 11A-11C show that ASO-mediated PARN knockdown leads to p53 accumulation and loss of viability in Hela cells. FIG. 11A shows representative western blots for PARN protein levels in Hela cells upon transfection with indicated ASOs (Average+/−S.D. for five independent replicates). FIG. 11B shows representative western blots for p53 levels upon treatment with Dox in ASO-transfected Hela cells (Average+/−S.D. for four independent replicates). FIG. 11C is a bar graph depicting viable Hela cells upon ASO transfection normalized to Control ASO transfected cells (Average+/−S.D. for three independent replicates).

FIGS. 12A and 12B show that PARN knockdown leads to p53 accumulation with or without DNA damage in HCT116 cells. FIG. 12A shows representative western blots for p53 and PARN protein levels in HCT cells transfected with Scr or PARN siRNA. FIG. 12B shows representative western blots for p53 levels upon Dox or EP treatment in PARN knockdown HCT116 cells (Average+/−S.D. for four biological replicates).

FIGS. 13A and 13B show that PARN knockdown leads to p53 accumulation with or without DNA damage in U87 glioblastoma cells. FIG. 13A shows representative western blots for p53 and PARN protein levels in Scr or PARN siRNA transfected U87 cells. FIG. 13B shows representative western blots for p53 levels in Dox or EP treated PARN knockdown U87 cells compared to control cells (Average+/−S.D. for five biological replicates for Dox treatment and three biological replicates for EP treatment).

### MODE(S) FOR CARRYING OUT THE INVENTION(S)

As used throughout this specification and the appended claims, the following terms have the following specified meaning. By “antisense,” as used herein in reference to nucleic acids, is meant a nucleic acid sequence, regardless of length, that is complementary to the coding strand of a gene. By “binding to” a molecule is meant having a physicochemical affinity for that molecule. For example, an antibody molecule may have affinity for an epitope found in a target protein. By “cancer” is meant a disease characterized by the pathological proliferation of a cell or tissue and its subsequent migration to or invasion of other tissues or organs. Cancer growth is typically uncontrolled and progressive, and occurs under conditions that would not elicit, or would cause cessation of, multiplication of normal cells. Cancers can affect a variety of cell types, tissues, or organs, including but not limited to an organ selected from the group consisting of bladder, bone, brain, breast, cartilage, glia, esophagus, fallopian tube, gallbladder, heart, intestines, kidney, liver, lung, lymph node, nervous tissue, ovaries, pancreas, prostate, skeletal muscle, skin, spinal cord, spleen, stomach, testes, thymus, thyroid, trachea, urogenital tract, ureter, urethra, uterus, and vagina, or a tissue or cell type thereof. Non-limiting examples of cancers include: acoustic neuroma, acute leukemia, acute lymphocytic leukemia, acute monocytic leukemia, acute myeloblastic leukemia, acute myelocytic leukemia, acute myelomonocytic leukemia, acute promyelocytic leukemia, acute erythroleukemia, adenocarcinoma, angiosarcoma, astrocytoma, basal cell carcinoma, bile duct carcinoma, bladder carcinoma, brain cancer, breast cancer, bronchogenic carcinoma, cervical cancer, chondrosarcoma, chordoma, choriocarcinoma, chronic leukemia, chronic lymphocytic leukemia, chronic myelocytic leukemia, colon cancer, colon carcinoma, craniopharyngioma, cystadenocarcinoma, embryonal carcinoma, endotheliosarcoma, ependymoma, epithelial carcinoma, Ewing's tumor, glioma, heavy chain disease, hemangioblastoma, hepatoma, Hodgkin's disease, large cell carcinoma, leiomyosarcoma, liposarcoma, lung cancer, lung carcinoma, lymphangioendotheliosarcoma, lymphangiosarcoma, macroglobulinemia, medullary carcinoma, medulloblastoma, melanoma, meningioma, mesothelioma, myxosarcoma, neuroblastoma, non-Hodgkin's disease, oligodendroglioma, osteogenic sarcoma, ovarian cancer, pancreatic cancer, papillary adenocarcinomas, papillary carcinoma, pincaloma, polycythemia vera, prostate cancer, rhabdomyosarcoma, renal cell carcinoma, retinoblastoma, schwannoma, sebaceous gland carcinoma, seminoma, small cell lung carcinoma, squamous cell carcinoma, sweat gland carcinoma, synovioma, testicular cancer, uterine cancer, Waldenstrom's fibrosarcoma, and Wilm's tumor.

By “chemotherapeutic agent” is meant one or more chemical agents used in the treatment or control of proliferative diseases (e.g., cancer). Chemotherapeutic agents include cytotoxic and cytostatic agents. Exemplary chemotherapeutic agents may mediate DNA damage (e.g., alkylating chemotherapeutic agents). Non-limiting examples of chemotherapeutic agents are described herein and are known in the art.

By “control sample” is meant a cell, cell sample, or protein or DNA sample that is used as a reference. For example, in experiments to determine activation of the p53 signaling pathway, the control sample may be a non-cancer cell (e.g., a non-cancer cell from a patient) or a cell that is not treated a genotoxic agent (e.g., a DNA-damaging chemotherapeutic agent), or a lysate prepared from such a cell. In experiments to determine inactivation of the p53 signaling pathway, the control sample may be a cell that has been treated with a genotoxic agent (e.g., a DNA-damaging chemotherapeutic agent).

By “detectably-labeled” is meant any means for marking and identifying the presence of a target molecule in a cell or a cell lysate. For example, antibodies or antisense nucleic acid molecules that recognize a target protein (e.g., PARN or p53 protein), mRNA (e.g., a PARN or p53 mRNA), or genomic DNA (e.g., gene encoding wild type, mutant, or truncated p53) in a cell or cell lysate may be detectably-labeled. Methods for detectably-labeling a molecule are well known in the art and include, without limitation, radionuclides (e.g., with an isotope such as 32P, 33P, 125I, or 35S), nonradioactive labeling (e.g., chemiluminescent labeling or fluorescein labeling), and epitope tags.

By “genotoxic agent” is meant any agent that causes, directly or indirectly, DNA damage in a cell. Non-limiting examples of genotoxic agents include DNA-damaging chemotherapeutic agents (e.g., doxorubicin), intercalating agents, UV light, and alkylating agents. Additional examples of genotoxic agents are known in the art.

By “hydrophobic” in the context of amino acids is meant any of the following amino acids: alanine, cysteine, isoleucine, leucine, methionine, phenylalanine, proline, tryptophan, tyrosine, or valine.

By “nucleic acid” is meant an oligomer or polymer of ribonucleic acid or deoxyribonucleic acid, or analog thereof. This term includes oligomers consisting of naturally occurring bases, sugars, and intersugar (backbone) linkages as well as oligomers having non-naturally occurring portions which function similarly. Such modified or substituted oligonucleotides are often preferred over native forms because of properties such as, for example, enhanced cellular uptake and increased stability in the presence of nucleases.

Specific examples of nucleic acids may contain phosphorothioates, phosphotriesters, methyl phosphonates, short chain alkyl or cycloalkyl intersugar linkages or short chain heteroatomic or heterocyclic intersugar linkages. Most preferred are those with CH₂—NH—O—CH₂, CH₂—N(CH₃)—O—CH₂, CH₂—O—N(CH₃)—CH₂, CH₂—N(CH₃)—N(CH₃)—CH₂ and O—N(CH₃)—CH₂—CH₂ backbones (where phosphodiester is O—P—O—CH₂). Also preferred are oligonucleotides having morpholino backbone structures (Summerton, J. E. and Weller, D. D., U.S. Pat. No. 5,034,506). In other preferred embodiments, such as the protein-nucleic acid (PNA) backbone, the phosphodiester backbone of the oligonucleotide may be replaced with a polyamide backbone, the bases being bound directly or indirectly to the aza nitrogen atoms of the polyamide backbone (P. E. Nielsen et al. Science 199:254, 1997). Other preferred oligonucleotides may contain alkyl and halogen-substituted sugar moieties comprising one of the following at the 2′ position: OH, SH, SCH3, F, OCN, O(CH2)nNH2 or O(CH2)n CH3, where n is from 1 to about 10; C1 to C10 lower alkyl, substituted lower alkyl, alkaryl or aralkyl; Cl; Br; CN; CF3; OCF3; O-, S-, or N-alkyl; O-, S-, or N-alkenyl; SOCH3; SO2CH3; ONO2; NO2; N3; NH2; heterocycloalkyl; heterocycloalkaryl; aminoalkylamino; polyalkylamino; substituted silyl; an RNA cleaving group; a conjugate; a reporter group; an intercalator; a group for improving the pharmacokinetic properties of an oligonucleotide; or a group for improving the pharmacodynamic properties of an oligonucleotide and other substituents having similar properties. Oligonucleotides may also have sugar mimetics such as cyclobutyls in place of the pentofuranosyl group.

Other preferred embodiments may include at least one modified base form. Some specific examples of such modified bases include 2-(amino) adenine, 2-(methylamino) adenine, 2-(imidazolylalkyl) adenine, 2-(aminoalklyamino) adenine, or other heterosubstituted alkyladenines.

By “p53 levels” or “p53 expression” is meant the amount of p53 protein or p53 mRNA present in a cell (e.g., a cancer cell or a control cell).

By “p53 protein” is meant a protein that is substantially identical to all or a part of any one of NCBI Accession Nos. BAC16799.1 (SEQ ID NO: 5), AAC12971.1 (SEQ ID NO: 6), P04637.4 (SEQ ID NO: 7), NP-000537.3 (SEQ ID NO: 8), NP-001119584.1 (SEQ ID NO: 9), AAD28535.1 (SEQ ID NO: 10), and AAD28628.1 (SEQ ID NO: 11).

By “p53 mRNA” is meant an mRNA that encodes a protein that is substantially identical to all or a part of any one of NCBI Accession Nos. BAC16799.1 (SEQ ID NO: 5), AAC12971.1 (SEQ ID NO: 6), P04637.4 (SEQ ID NO: 7), NP-000537.3 (SEQ ID NO: 8), NP-001119584.1 (SEQ ID NO: 9), AAD28535.1 (SEQ ID NO: 10), and AAD28628.1 (SEQ ID NO: 11).

By “p53 gene” or “p53 genomic DNA” is meant a sequence of genomic DNA that encodes a wild type, mutant, or truncated p53 protein that encodes a protein that is substantially identical to all or a part of (e.g., at least 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, or 390 amino acids) any one of NCBI Accession Nos. BAC16799.1 (SEQ ID NO: 68), AAC12971.1 (SEQ ID NO: 69), P04637.4 (SEQ ID NO: 70), NP-000537.3 (SEQ ID NO: 71), NP-001119584.1 (SEQ ID NO: 72), AAD28535.1 (SEQ ID NO: 73), and AAD28628.1 (SEQ ID NO: 74). For example, a mutant p53 gene may encode a p53 protein that contains at one or more (e.g., at least two, three, four, five, six, seven, eight, nine, or ten) amino acid substitutions, deletions, and/or additions. A mutant p53 gene may encode a p53 protein that contains at least a 5 amino acid truncation (e.g., at least a 10, 15, 20, 25, 30, 35, 40, 45, 50, 60, 70, 80, 90, 100, 110, 120, 130, 140, 150, 160, 170, 180, 190, or 200 amino acid truncation) or at least a 5 amino acid addition (e.g., at least a 10, 15, 20, 25, 30, 35, 40, 45, 50, 60, 70, 80, 90, 100, 110, 120, 130, 140, 150, 160, 170, 180, 190, or 200 amino acid addition) (e.g., a fusion protein resulting from a gene translocation).

By “mutant or truncated p53 with reduced expression or activity” is meant a p53 protein that contains at least one amino acid substitution, deletion, and/or addition compared to the wild type sequence of p53 protein (or an mRNA encoding such a p53 protein) that results in a decrease in expression of p53 protein or a decrease in p53 activity in the cell. For example, a mutant p53 protein may contain one or more (e.g., at least two, three, four, five, six, seven, eight, nine, or ten) amino acid substitutions, deletions, and/or additions (e.g., a fusion protein resulting from a gene translocation) that decreases the ability of p53 to bind to DNA, mediate cell cycle arrest in response to genotoxic stress, and/or stimulate p21 gene expression. A mutant p53 protein may contain at least a 5 amino acid truncation (e.g., at least a 10, 15, 20, 25, 30, 35, 40, 45, 50, 60, 70, 80, 90, 100, 110, 120, 130, 140, 150, 160, 170, 180, 190, or 200 amino acid truncation) or at least a 5 amino acid addition (e.g., at least a 10, 15, 20, 25, 30, 35, 40, 45, 50, 60, 70, 80, 90, 100, 110, 120, 130, 140, 150, 160, 170, 180, 190, or 200 amino acid addition) (e.g., a fusion protein resulting from a gene translocation) compared to the wild type p53 protein. Several examples of mutant or truncated p53 are known in the art. In addition, a mutant p53 protein may result from a mutation in one or both alleles of a p53 gene. For example, a mutation in the second allele of a p53 gene may be detected in a cell having a mutation in the first allele of a p53 gene (e.g., a loss of heterozygosity mutation).

By “p53 activity” is meant an activity of wild type p53 protein in a cell. Non-limiting examples of p53 activity include DNA-binding activity, ability to mediate cell cycle arrest, and induction of p21 gene expression. Assays for measuring in vitro and in vivo p53 activity are known in the art.

By “pharmaceutically acceptable excipient” is meant a carrier that is physiologically acceptable to the subject to which it is administered and that preserves the therapeutic properties of the compound with which it is administered. One exemplary pharmaceutically acceptable excipient is physiological saline. Other physiologically acceptable excipients and their formulations are known to one skilled in the art and described, for example, in “Remington: The Science and Practice of Pharmacy,” (20th ed., ed. A. R. Gennaro, 2000, Lippincott Williams & Wilkins).

By “prodrug” is meant a compound that is modified in vivo, resulting in formation of a biologically active drug compound, for example by hydrolysis in blood. A thorough discussion of prodrug modifications is provided in T. Higuchi and V. Stella, Pro-drugs as Novel Delivery Systems, Vol. 14 of the A.C.S. Symposium Series, Edward B. Roche, Ed., Bioreversible Carriers in Drug Design, American Pharmaceutical Association and Pergamon Press, 1987, and Judkins et al., Synthetic Communications 26 (23): 4351-4367, 1996, each of which is incorporated herein by reference.

By “poly (A)-specific ribonuclease” or “PARN” is meant a protein substantially identical to any one of NCBI Accession Nos. NP-002573.1 (SEQ ID NO: 1), AAH50029.1 (SEQ ID NO: 2), 095453.1 (SEQ ID NO: 3), and CAA06683.1 (SEQ ID NO: 4), or a nucleic acid encoding a protein substantially identical to any one of NCBI Accession Nos. NP-002573.1 (SEQ ID NO: 1), AAH50029.1 (SEQ ID NO: 2), 095453.1 (SEQ ID NO: 3), and CAA06683.1 (SEQ ID NO: 4).

By “phosphorylated PARN” is meant a PARN protein that has been phosphorylated. For example, the term phosphorylated PARN includes a PARN protein that is phosphorylated at serine-557.

By “reducing the severity of one or more symptoms” is meant a reduction (e.g., at least 5%, 10%, 15%, 20%, 25%, 30%, 35%, 40%, 45%, 50%, 55%, 60%, 65%, 70%, 75%, 80%, 85%, 90%, 95%, or 100%) in the severity or duration of at least one (e.g., at least two, three, four, five, or six) symptoms of a disease (e.g., a cancer). For example, the methods of the invention may result in at a 10% reduction in at least one (e.g., at least two, three, four, five, or six) symptoms of cancer.

By “RNA interference” (RNAi) is meant a phenomenon where double-stranded RNA homologous to a target mRNA leads to degradation of the targeted mRNA (e.g., a PARN mRNA). RNAi is more broadly defined as degradation of target mRNAs by homologous siRNAs.

By “siNA” is meant small interfering nucleic acids. One exemplary siNA is composed of ribonucleic acid (siRNA). siRNAs can be 21-25 nt RNAs derived from processing of linear double-stranded RNA. siRNAs assemble in complexes termed RISC (RNA-induced silencing complex) and target homologous RNA sequences for endonucleolytic cleavage. Synthetic siRNAs also recruit RISCs and are capable of cleaving homologous RNA sequences.

By “substantially identical” is meant a polypeptide or nucleic acid exhibiting at least 75%, 80%, 85%, 90%, 91%, 92%, 93%, 94, 95%, 96%, 97%, 98%, 99%, or even 100% identity to a reference amino acid or nucleic acid sequence. For polypeptides, the length of comparison sequences will generally be at least 35 amino acids, 45 amino acids, 55 amino acids, or even 70 amino acids. For nucleic acids, the length of comparison sequences will generally be at least 60 nucleotides, 90 nucleotides, or even 120 nucleotides.

Sequence identity is typically measured using publicly available computer programs. Computer program methods to determine identity between two sequences include, but are not limited to, the GCG program package (Devereux et al., Nucleic Acids Research 12:387, 1984), BLASTP, BLASTN, and FASTA (Altschul et al., J. Mol. Biol. 215:403, 1990). The well-known Smith Waterman algorithm may also be used to determine identity. The BLAST program is publicly available from NCBI and other sources (e.g., BLAST Manual, Altschul et al., NCBI NLM NIH, Bethesda, Md. 20894). These software programs match similar sequences by assigning degrees of homology to various substitutions, deletions, and other modifications. Conservative substitutions for amino acid comparisons typically include substitutions within the following groups: glycine, alanine, valine, isoleucine, leucine; aspartic acid, glutamic acid, asparagine, glutamine; serine, threonine; lysine, arginine; and phenylalanine, tyrosine.

By the term “symptoms of cancer” is meant one or more (e.g., one, two, three, four, or five) of the physical manifestations of cancer. Non-limiting examples of symptoms of cancer include blood in urine, pain or burning upon urination, cloudy urine, pain in bone, fractures in bones, fatigue, weight loss, repeated infections, nausea, vomiting, constipation, numbness in the legs, bruising, dizziness, drowsiness, abnormal eye movements, changes in vision, changes in speech, headaches, thickening of a tissue, rectal bleeding, abdominal cramps, loss of appetite, fever, enlarged lymphnodes, persistent cough, blood in sputum, lung congestion, itchy skin, lumps in skin, abdominal swelling, vaginal bleeding, jaundice, heartburn, indigestion, cell proliferation, and loss of regulation of controlled cell death.

By “treating” a disease, disorder, or condition is meant delaying an initial or subsequent occurrence of a disease, disorder, or condition; increasing the disease-free survival time between the disappearance of a condition and its reoccurrence; stabilizing or reducing one or more (e.g., two, three, four, or five) adverse symptom(s) associated with a condition; or inhibiting, slowing, or stabilizing the progression of a condition. The term “treating” also includes reducing (e.g., by at least 5%, 10%, 15%, 20%, 25%, 30%, 35%, 40%, 45%, 50%, 55%, 60%, 65%, 70%, 75%, 80%, 85%, 90%, or 95% the severity or duration of one or more (e.g., one, two, three, four, or five) symptoms of a disease (e.g., cancer) in a patient. Desirably, at least 20%, 40%, 60%, 80%, 90%, or 95% of the treated subjects have a complete remission in which all evidence of the disease disappears. In another desirable embodiment, the length of time a patient survives after being diagnosed with a condition and treated using the methods of the invention is at least 20%, 40%, 60%, 80%, 100%, 200%, or even 500% greater than (i) the average amount of time an untreated patient survives or (ii) the average amount of time a patient treated with another therapy survives.

In view of the characterization of the RNA regulatory effects of PARN activity and its importance for maintenance of p53 tumor suppression, this disclosure provides methods of reducing the severity of one or more symptoms of cancer in a patient, methods of identifying a cancer patient that may selectively benefit from the administration of one or more PARN inhibitor(s) or the combination of one or more PARN inhibitor(s) and one or more chemotherapeutic agent(s), methods of identifying a cancer patient that may selectively benefit from the administration of one or more chemotherapeutic agent(s), methods of diagnosing a chemotherapy-resistant cancer or a chemotherapy-sensitive cancer cell in a patient, and kits for diagnosing a chemotherapy-resistant or chemotherapy-sensitive cancer in a patient.

This disclosure provides methods for treating cancer that include a step for determining the activation or inactivation of the PARN exonuclease in cancer cell(s) from the patient and, optionally, determining the inactivation of the p53 pathway in cancer cell(s) from the patient. In view of this determination(s), the patient may be differentially administered one or more PARN inhibitor(s) or the combination of one or more PARN inhibitor(s) and one or more chemotherapeutic agent(s) or administered one or more chemotherapeutic agent(s). This disclosure further provides methods of treating a cancer patient diagnosed as having a chemotherapy-resistant or a chemotherapy-sensitive cancer using the diagnostic methods provided herein (e.g., by a diagnostic or clinical laboratory), where a patient diagnosed as having a chemotherapy-resistant cancer is administered one or more PARN inhibitor(s) and a patient diagnosed as having a chemotherapy-sensitive cancer is administered one or more chemotherapeutic agent(s).

The inventors have characterized the PARN exonuclease activity that is required for the maintenance of the p53 pathway. They have demonstrated that PARN stabilizes mature and precursor miRNAs by removing oligo (A) tails added by the poly (A) polymerase PAPD5, thereby preventing the exonucleases DIS3L or DIS3L2 to degrade the miRNAs. PARN-regulated miRNAs affect multiple cellular processes, and several downregulated miRNAs are negative regulators of the p53 tumor suppressor protein, which is upregulated in patients with PARN deficiency. They have also demonstrated that PARN knockdown destabilizes multiple miRNAs that repress p53 expression, which leads to an increase in p53 accumulation in a Dicer-dependent manner, thus explaining why PARN defective patients show p53 accumulation.

PARN exonuclease inhibition may be indicated by one or more (e.g., two, three, four, five, or six) of the following features: decreased (e.g., by at least 10%, 20%, 30%, 40%, 50%, 60%, 70%, 80%, or 90%) PARN protein in the cytoplasm, decreased (e.g., by at least 10%, 20%, 30%, 40%, 50%, 60%, 70%, 80%, or 90%) PARN protein in the nucleus, and decreased (by at least 10%, 20%, 30%, 40%, 50%, 60%, 70%, 80%, or 90%) levels of phosphorylated PARN (e.g., phosphorylation at serine 557).

The amount of PARN protein in the cytoplasm or the nucleus of a cell may be measured using an antibody that is specific for PARN. In one example, a cell may be differentially lysed to prepare a separate nuclear extract and/or cytosolic lysates. Immunoblotting may be performed using an PARN antibody to determine the levels of PARN protein found in the cytoplasm and/or the nucleus. Alternatively, the relative amount of PARN in the nucleus and cytoplasm may be measured by immunofluorescence microscopy using labeled PARN antibodies (e.g., fluorescently-labeled antibodies). The relative increase in PARN protein levels in the cytoplasm or the relative decrease in PARN protein levels in the nucleus of a cancer cell may be compared to a non-cancerous cell (e.g., a non-cancerous cell from the patient), or a cell that has been exposed to a genotoxic agent (e.g., a DNA-damaging or chemotherapeutic agent). The relative decrease in PARN protein levels in the cytoplasm or the relative increase in PARN protein levels in the nucleus of a cancer cell may be compared to a cancer cell or a cell that has been exposed to a genotoxic agent.

The total amount of phosphorylated PARN protein may be measured using methods known in the art. Such techniques often utilize an antibody that specifically recognizes the phosphorylated form of PARN protein. In one example, a cellular lysate from cancer cells may be prepared and immunoblotted using an antibody that specifically binds phosphorylated PARN. Alternatively, the total amount of phosphorylated PARN present in a cell may be measured using immunofluorescent microscopy or fluorescence-assisted cell sorting (FACS) that utilizes a fluorescently-labeled antibody that specifically binds to phosphorylated PARN. Similarly, the amount of phosphorylated PARN in the cytoplasm or nucleus may be measured using antibodies that specifically bind to the phosphorylated form of PARN. For example, a cytosolic extract or nuclear extract may be prepared from cancer cells using differential lysis and the prepared extract immunoblotted using an antibody that specifically binds to phosphorylated PARN. Similarly, immunofluorescence microscopy may be performed using a fluorescently-labeled antibody that specifically binds to phosphorylated PARN to measure the amount of phosphorylated PARN that is present in a cancer cell (e.g., the amount of phosphorylated PARN protein that is present in the cytosol or nucleus).

The relative increase in total phosphorylated PARN protein or the relative increase in phosphorylated PARN protein in the cytoplasm or nucleus of a cancer cell(s) may be compared to a non-cancerous cell (e.g., a non-cancerous cell from the patient) or a cell that has not been exposed to a genotoxic agent (e.g., a DNA-damaging or chemotherapeutic agent). The relative decrease in total phosphorylated PARN protein or the relative decrease in phosphorylated PARN protein in the cytoplasm or nucleus of a cancer cell(s) may be compared to a cancer cell or a cell that has been exposed to a genotoxic agent.

The phosphorylation of PARN (e.g., phosphorylation at serine 557), may also be measured using antibodies that specifically recognize the phosphorylated forms of PARN. As described above, antibodies that specifically bind to the phosphorylated form of PARN may be used to measure the total amount of the phosphorylated protein present in a cell or a cell extract. For example, these phosphorylation-specific antibodies may be used to perform immunoblotting on extracts prepared from cancer cell(s). Such methods may be automated or performed using protein chip assays. Alternatively, phosphosphorylation-specific antibodies may be fluorescently-labeled and used in FACS analysis or immunofluorescent microscopy to measure the total amount of the target phosphorylated protein present in a cancer cell. The relative increase in phosphorylated PARN may be compared to a non-cancerous cell (e.g., a non-cancerous cell from the patient) or a cell that has not been exposed to a genotoxic agent (e.g., a DNA-damaging or chemotherapeutic agent). The relative decrease in phosphorylated PARN may be compared to a cancer cell or a cell that has been exposed to a genotoxic agent.

The p53 pathway has been shown to mediate cell cycle arrest, and p53 induction is associated with growth arrest and apoptosis. Specifically, in cancer cells, a mutation or truncation (e.g., one or more amino acid substitutions, deletions, and/or additions) of the p53 protein results in decreased activity or expression, resulting for example, in a decrease in DNA-binding activity, a decrease in the ability to induce p21 induction, or a decrease in the ability to mediate cell cycle arrest in response to genotoxic stress. Similarly, inactivation of p53 may occur in the cell by way of a gene translocation event which results in the formation of a p53 fusion protein. Further, inactivation of p53 may occur by a loss of heterozygosity mutation, where the mutation in a second allele of the p53 gene occurs following a mutation in the first allele of the p53 gene. Thus, loss of p53 signaling may be indicated by one or more (e.g., two, three, or four) of the following features: decreased (e.g., by at least 10%, 20%, 30%, 40%, 50%, 60%, 70%, 80%, or 90%) p53 mRNA or protein levels, expression of a mutant or truncated p53 with decreased (e.g., by at least 10%, 20%, 30%, 40%, 50%, 60%, 70%, 80%, or 90%) expression or activity, and decreased (by at least 10%, 20%, 30%, 40%, 50%, 60%, 70%, 80%, or 90%) p21 expression or activity. Various methods for measuring p53 pathway inactivation are known in the art and non-limiting examples are provided below.

The levels of p53 mRNA or protein may be measured using a number of molecular biology techniques known in the art. p53 mRNA may be measured using any nucleic acid that is complementary to a contiguous sequence present in p53 mRNA. For example, the amount of p53 mRNA may be detected using FISH using such an antisense nucleic acid. p53 mRNA levels may also be measured using techniques based on PCR using primers specifically designed to amplify an mRNA encoding p53 protein (e.g., reverse-transcriptase PCR, real-time qPCR, or gene array technology). p53 protein levels may be measured using an antibody that specific binds to p53 protein. For example, immunoblotting may be performed on whole cell extract using a p53 antibody. Similarly, a fluorescently-labeled p53 antibody may be used to perform immunofluorescence microscopy or FACS analysis on cancer cells. The relative decrease in p53 protein or mRNA levels may be compared to a non-cancerous cell (e.g., a non-cancerous cell from the patient). Importantly, measurements of p21 protein expression by immunoblotting, immunohistochemistry, or immunofluorescence microscopy, for example, may be used as highly sensitive assays of p53 function.

The expression of a mutant or truncated p53 protein with decreased expression or activity (e.g., decreased DNA-binding activity, ability to induce cell cycle arrest following genotoxic stress, and/or the ability to induce p21 gene expression) may be measured using molecular biology techniques known in the art. For example, mutations or truncations in p53 protein may be detected using PCR-based techniques using primers that specifically amplify the region of the p53 mRNA or gene (e.g., reverse-transcriptase PCR, real-time qPCR, or gene array technology). In addition, methods to analyze or determine the presence of a mutation in a second allele of the p53 locus may be identified using single-nucleotide polymormorphism microarray analysis.

Inactivated p53 signaling may also be observed by a decrease in p21 mRNA or protein expression in a cell (e.g., reduced induction of p21 expression following genotoxic stress). p53 mRNA may be may be measured using any nucleic acid that is complementary to a contiguous sequence present in p53 mRNA. For example, the amount of p21 mRNA may be detected using FISH using such an antisense nucleic acid. p21 mRNA levels may also be measured using techniques based on PCR using primers specifically designed to amplify an mRNA encoding p21 protein (e.g., reverse-transcriptase PCR, real-time qPCR, or gene array technology). p21 protein levels may be measured using an antibody that specific binds to p21 protein. For example, immunoblotting may be performed on whole cell extract using a p21 antibody. Similarly, a fluorescently-labeled p21 antibody may be used to perform immunofluorescence microscopy or FACS analysis on cancer cells. The relative decrease in p21 protein or mRNA levels may be compared to a non-cancerous cell (e.g., a non-cancerous cell from the patient or a non-cancerous cell exposed to a genotoxic agent, such as a DNA-damaging chemotherapeutic agent).

Any compound or pharmaceutical composition that inhibits an activity of PARN may be useful in the methods of treatment provided by this disclosure. Non-limiting examples of PARN inhibitors are described below.

### Peptides

Peptides that mimic a natural peptide substrate of PARN may decrease the extent or rate with which PARN is able to bind to its natural substrates in vivo. Accordingly, such peptides may be used as PARN inhibitors in the treatment methods provided by this disclosure.

### Small Molecules

Any small molecule that inhibits PARN (e.g., PARN deadenylase activity), whether specifically or nonspecifically, may be of utility in the methods provided by this disclosure. Other small molecule inhibitors of PARN are described in Balatsos, et al., Biochemistry 2009, 48:6044-51.

PARN antisense nucleic acids may be also be used as PARN inhibitors in the methods of this disclosure. Sequence-specific suppression of gene expression can be achieved by intracellular hybridization between mRNA and a complementary antisense species. The formation of a hybrid RNA duplex may then interfere with the processing/transport/translation and/or stability of the target PARN mRNA. Antisense strategies may use a variety of approaches, including the use of antisense oligonucleotides and injection of antisense RNA. An exemplary approach features transfection of antisense RNA expression vectors into targeted cells. Antisense effects can be induced by control (sense) sequences; however, the extent of phenotypic changes are highly variable. Phenotypic effects induced by antisense effects are based on changes in criteria such as protein levels, protein activity measurement, and target mRNA levels.

Computer programs such as OLIGO (previously distributed by National Biosciences Inc.) may be used to select candidate nucleobase oligomers for antisense therapy based on the following criteria:
1) No more than 75% GC content, and no more than 75% AT content;
2) Preferably no nucleobase oligomers with four or more consecutive G residues (due to reported toxic effects, although one was chosen as a toxicity control);
3) No nucleobase oligomers with the ability to form stable dimers or hairpin structures; and
4) Sequences around the translation start site are a preferred region.

In addition, accessible regions of the target mRNA may be predicted with the help of the RNA secondary structure folding program MFOLD (M. Zuker, D. H. Mathews & D. H. Turner, Algorithms and Thermodynamics for RNA Secondary Structure Prediction: A Practical Guide. In: RNA Biochemistry and Biotechnology, J. Barciszewski & B. F. C. Clark, eds., NATO ASI Series, Kluwer Academic Publishers, 1999). Sub-optimal folds with a free energy value within 5% of the predicted most stable fold of the mRNA may be predicted using a window of 200 bases within which a residue can find a complimentary base to form a base pair bond. Open regions that do not form a base pair may be summed together with each suboptimal fold, and areas that consistently are predicted as open may be considered more accessible to the binding to nucleobase oligomers. Additional nucleobase oligomer that only partially fulfill some of the above selection criteria may also be chosen as possible candidates if they recognize a predicted open region of the target mRNA.

Nucleobase oligomers may be used as PARN inhibitors in the methods of this disclosure. For example, double-stranded RNAs may be used to elicit RNAi-mediated knockdown of PARN expression. RNAi is a method for decreasing the cellular expression of specific proteins of interest (reviewed in Tuschl, Chembiochem 2:239-245, 2001; Sharp, Genes & Devel. 15:485-490, 2000; Hutvagner and Zamore, Curr. Opin. Genet. Devel. 12:225-232, 2002; and Hannon, Nature 418:244-251, 2002). In RNAi, gene silencing is typically triggered post-transcriptionally by the presence of double-stranded RNA (dsRNA) in a cell. This dsRNA is processed intracellularly into shorter pieces called small interfering RNAs (siRNAs). The introduction of siRNAs into cells either by transfection of dsRNAs or through expression of siRNAs using a plasmid-based expression system is increasingly being used to create loss-of-function phenotypes in mammalian cells.

In one embodiment of this disclosure, a double-stranded RNA (dsRNA) molecule is made. The dsRNA can be two distinct strands of RNA that have duplexed, or a single RNA strand that has self-duplexed (small hairpin (sh) RNA). Typically, dsRNAs are about 21 or 22 base pairs, but may be shorter or longer (up to about 29 nucleobases) if desired. dsRNA can be made using standard techniques (e.g., chemical synthesis or in vitro transcription). Kits are available, for example, from Ambion (Austin, Tex.) and Epicentre (Madison, Wis.). Methods for expressing dsRNA in mammalian cells are described in Brummelkamp et al. Science 296:550-553, 2002; Paddison et al. Genes & Devel. 16:948-958, 2002. Paul et al. Nature Biotechnol. 20:505-508, 2002; Sui et al. Proc. Natl. Acad. Sci. USA 99:5515-5520, 2002; Yu et al. Proc. Natl. Acad. Sci. USA 99:6047-6052, 2002; Miyagishi et al. Nature Biotechnol. 20:497-500, 2002; and Lee et al. Nature Biotechnol. 20:500-505 2002.

Small hairpin RNAs consist of a stem-loop structure with optional 3′ UU-overhangs. While there may be variation, stems can range from twenty-one to thirty-one base pairs (desirably twenty-five to twenty-nine base pairs), and the loops can range from four to thirty base pairs (desirably four to twenty-three base pairs). For expression of shRNAs within cells, plasmid vectors containing, e.g., the polymerase III H1-RNA or U6 promoter, a cloning site for the stem-looped RNA insert, and a 4-5-thymidine transcription termination signal can be employed. The Polymerase III promoters generally have well-defined initiation and stop sites and their transcripts lack poly (A) tails. The termination signal for these promoters is defined by the polythymidine tract, and the transcript is typically cleaved after the second uridine. Cleavage at this position generates a 3′ UU overhang in the expressed shRNA, which is similar to the 3′ overhangs of synthetic siRNAs. Additional methods for expressing the shRNA in mammalian cells are described in the references cited above.

Computer programs that employ rational design of oligos are useful in predicting regions of the PARN sequence that may be targeted by RNAi. For example, see Reynolds et al., Nat. Biotechnol., 22:326-330, 2004, for a description of the Dharmacon siDESIGN tool.

Additional PARN inhibitors include antibodies (e.g., human monoclonal antibodies) that specifically bind to total PARN or phosphorylated PARN, or functional fragments thereof. Methods for the generation of monoclonal antibodies using hybridoma technology are known in the art. PARN-specific antibodies are desirably produced using PARN protein sequences that do not reside within highly conserved regions, and that appear likely to be antigenic, as evaluated by criteria such as those provided by the Peptide Structure Program (Genetics Computer Group Sequence Analysis Package, Program Manual for the GCG Package, Version 7, 1991) using the algorithm of Jameson et al., CABIOS 4:181, 1988. These fragments can be generated by standard techniques, e.g., by PCR, and cloned into any appropriate expression vector. For example, GST fusion proteins can be expressed in E. coli and purified using a glutathione-agarose affinity matrix. To minimize the potential for obtaining antisera that is non-specific or exhibits low-affinity binding to PARN, two or three PARN fusion proteins may be generated for each fragment injected into a separate animal. Antisera are raised by injections in series, preferably including at least three booster injections.

In addition to intact monoclonal and polyclonal anti-PARN protein, various genetically engineered antibodies and antibody fragments (e.g., F(ab′)2, Fab′, Fab, Fv, and sFv fragments) can be produced using standard methods. Truncated versions of monoclonal antibodies, for example, can be produced by recombinant methods in which plasmids are generated that express the desired monoclonal antibody fragment(s) in a suitable host. Ladner (U.S. Pat. Nos. 4,946,778 and 4,704,692) describes methods for preparing single polypeptide chain antibodies. Ward et al., Nature 341:544-546, 1989, describes the preparation of heavy chain variable domain which have high antigen-binding affinities. McCafferty et al. (Nature 348:552-554, 1990) show that complete antibody V domains can be displayed on the surface of fd bacteriophage, that the phage bind specifically to antigen, and that rare phage (one in a million) can be isolated after affinity chromatography. Boss et al. (U.S. Pat. No. 4,816,397) describes various methods for producing immunoglobulins, and immunologically functional fragments thereof, that include at least the variable domains of the heavy and light chains in a single host cell. Cabilly et al. (U.S. Pat. No. 4,816,567) describes methods for preparing chimeric antibodies. In addition, the antibodies can be coupled to compounds, such as toxins or radiolabels.

As described above, the PARN inhibitor may be a small molecule, a peptide, or a nucleic acid molecule. In some instances, a compound that is effective in vitro in inhibiting PARN polypeptide is not an effective therapeutic agent in vivo. For example, this could be due to low bioavailability of the PARN inhibitor. One way to circumvent this difficulty is to administer a modified drug, or prodrug, with improved bioavailability that converts naturally to the original compound following administration. Such prodrugs may undergo transformation before exhibiting their full pharmacological effects. Prodrugs contain one or more specialized protective groups that are specifically designed to alter or to eliminate undesirable properties in the parent molecule. In one embodiment, a prodrug masks one or more charged or hydrophobic groups of a parent molecule. Once administered, a prodrug is metabolized in vivo into an active compound.

Prodrugs may be useful for improving one or more of the following characteristics of a drug: solubility, absorption, distribution, metabolization, excretion, site specificity, stability, patient acceptability, reduced toxicity, or problems of formulation. For example, an active compound may have poor oral bioavailability, but by attaching an appropriately-chosen covalent linkage that may be metabolized in the body, oral bioavailability may improve sufficiently to enable the prodrug to be administered orally without adversely affecting the parent compound's activity within the body.

A prodrug may be carrier-linked, meaning that it contains a group such as an ester that can be removed enzymatically. Optimally, the additional chemical group has little or no pharmacologic activity, and the bond connecting this group to the parent compound is labile to allow for efficient in vivo activation. Such a carrier group may be linked directly to the parent compound (bipartate), or it may be bonded via a linker region (tripartate). Common examples of chemical groups attached to parent compounds to form prodrugs include esters, methyl esters, sulfates, sulfonates, phosphates, alcohols, amides, imines, phenyl carbamates, and carbonyls.

As one example, methylprednisolone is a poorly water-soluble corticosteroid drug. In order to be useful for aqueous injection or ophthalmic administration, this drug must be converted into a prodrug of enhanced solubility. Methylprednisolone sodium succinate ester is much more soluble than the parent compound, and it is rapidly and extensively hydrolysed in vivo by cholinesterases to free methylprednisolone.

Caged compounds may also be used as prodrugs. A caged compound may have, e.g., one or more photolyzable chemical groups attached that renders the compound biologically inactive. In this example, flash photolysis releases the caging group (and activates the compound) in a spatially or temporally controlled manner. Caged compounds may be made or designed by any method known to those of skill in the art.

For further description of the design and use of prodrugs, see Testa and Mayer, Hydrolysis in Drug and Prodrug Metabolism: Chemistry, Biochemistry and Enzymology, published by Vch. Verlagsgesellschaft Mbh. (2003).

Other modified compounds are also possible in the methods of this disclosure. For example, a modified compound need not be metabolized to form a parent molecule. Rather, in some embodiments, a compound may contain a non-removable moiety that, e.g., increases bioavailability without substantially diminishing the activity of the parent molecule. Such a moiety could, for example, be covalently-linked to the parent molecule and could be capable of translocating across a biological membrane such as a cell membrane, in order to enhance cellular uptake. Exemplary moieties include peptides, e.g., penetratin or TAT. An exemplary penetratin-containing compound according to this disclosure is, e.g., a peptide comprising the sixteen amino acid sequence from the homeodomain of the Antennapedia protein (Derossi et al., J. Biol. Chem. 269:10444-10450, 1994) or including a peptide sequence disclosed by Lin et al. (J. Biol. Chem. 270:14255-14258, 1995). Others are described in U.S. Patent Application Publication No. 2004/0209797 and U.S. Pat. Nos. 5,804,604, 5,747,641, 5,674,980, 5,670,617, and 5,652,122. In addition, a compound of this disclosure could be attached, for example, to a solid support.

A cancer patient identified as having cancer cell(s) with an inactivated p53 pathway (e.g., cells with elevated or active PARN and an inactivated p53 pathway) may selectively benefit from the administration of one or more (e.g., two, three, four, or five) chemotherapeutic agent(s) relative to a patient having a cancer cell(s) with inhibited or inactivated PARN and/or p53 pathway. For example, cancer patients that are implicated as having a repressed p53 pathway may experience at least a 10% (e.g., at least 20%, 30%, 40%, 50%, 60%, 70%, 80%, or 90%) in one or more symptoms of cancer following treatment with one or more chemotherapeutic agents compared to a cancer subject having cancer cells with active or elevated PARN and an inactivated p53 pathway following treatment with the same chemotherapeutic agents. Based on the present inventor's discovery, a skilled physician may recommend to a patient having cancer cells with an inactivated p53 pathway (e.g., cancer cells with an elevated or active PARN and an inactivated p53 pathway), a therapeutic regime that includes the administration of an inhibitor of PARN and one or more chemotherapeutic agents (e.g. the administration of an additional dosage of a chemotherapeutic agent to a patient that has previously received a dosage of a chemotherapeutic agent). In addition, a cancer patient diagnosed as having a chemotherapy-sensitive cancer (e.g., by a diagnostic or clinical laboratory) using the diagnostic methods described herein, may be administered one or more chemotherapeutic agent(s) with or without the co-administration of a PARN inhibitor.

A variety of chemotherapeutic agents are known in the art. Desirably, the chemotherapeutic agent administered induces apoptosis or necrosis of the cancer cells. Non-limiting examples of chemotherapeutic agents useful in these methods include: alemtuzumab, altretamine, aminoglutethimide, amsacrine, anastrozole, azacitidine, bleomycin, bicalutamide, busulfan, capecitabine, carboplatin, carmustine, celecoxib, chlorambucil, 2-chlorodeoxyadenosine, cisplatin, colchicine, cyclophosphamide, cytarabine, cytoxan, dacarbazine, dactinomycin, daunorubicin, docetaxel, doxorubicin, epirubicin, estramustine phosphate, etodolac, etoposide, exemestane, floxuridine, fludarabine, 5-fluorouracil, flutamide, formestane, gemcitabine, gentuzumab, goserelin, hexamethylmelamine, hydroxyurea, hypericin, ifosfamide, imatinib, interferon, irinotecan, letrozole, leuporelin, lomustine, mechlorethamine, melphalen, mercaptopurine, 6-mercaptopurine, methotrexate, mitomycin, mitotane, mitoxantrone, nilutamide, nocodazole, paclitaxel, pentostatin, procarbazine, raltitrexed, rituximab, rofecoxib, streptozocin, tamoxifen, temozolomide, teniposide, 6-thioguanine, topotecan, toremofine, trastuzumab, vinblastine, vincristine, vindesine, and vinorelbine.

### Administration Schedules and Formulations

The methods of treatment provided by this disclosure may require the steps of determining the activity or expression level of PARN and, optionally, steps of determining the activity or inactivation of the p53 signaling pathway. Upon a determination that a cancer patient has an active or elevated level of PARN and/or an inactive p53 pathway, the patient is administered one or more dosages (e.g., at least two, three, four, five, six, seven, eight, nine, or ten dosages) of a one or more (e.g., two, three, four, or five) chemotherapeutic agents. Upon a determination that a cancer patient has an active or elevated level of PARN and/or an inactive p53 pathway, the patient is administered one or more dosages (e.g., at least two, three, four, five, six, seven, eight, nine, or ten dosages) or one or more (e.g., two, three, four, or five) PARN inhibitor(s). In certain embodiments of these methods, the determination of the level or activity of PARN and, optionally the determination of inactivation of the p53 pathway, is performed by a diagnostic or clinical laboratory. Following a determination that a cancer patient has active or elevate PARN (e.g., a cancer patient having active or elevated PARN and an inactivated p53 pathway), the patient is administered one or more dosages (e.g., at least two, three, four, five, six, seven, eight, nine, or ten dosages) of one or more (e.g., two, three, four, or five) PARN inhibitors or one or more dosages (e.g., at least two, three, four, five, six, seven, eight, nine, or ten dosages) of one or more (e.g., two, three, four, or five) PARN inhibitors and one or more (e.g., two, three, four, or five) chemotherapeutic agents.

In any of the methods described herein, each of the one or more PARN inhibitors may be administered in a dosage between 0.1 mg and 1 g. The specific dosage of each PARN inhibitor to be administered to the subject may vary depending upon the chemical nature of the PARN inhibitor. The PARN inhibitor(s) may be formulated for any known route of administration, including oral, intravenous, intraarterial, intraocular, intranasal, intramuscular, and subcutaneous administration. The PARN inhibitor may be administered to cancer patients once a day, twice a day, three times a day, once a week, twice a week, three times a week, four times a week, five times a week, six times a week, seven times a week, bi-weekly, tri-weckly, monthly, every two months, every three months, every four months, every five months, twice a year, three times a year, four times a year, five times a year, or six times a year. The specific dosage and administration schedule for a PARN inhibitor may be determined by a skilled physician based on a number of factors including the age, weight, and sex of the patient, the type of cancer, and the severity of one or more symptoms of cancer.

In any of the methods described herein, each of the one or more chemotherapeutic agents may be administered in a dosage between 0.1 mg and 1 g. The specific dosage of each chemotherapeutic agent to be administered to the subject may vary depending upon the chemical nature of the chemotherapeutic agent. The chemotherapeutic agent(s) may be formulated for any known route of administration, including oral, intravenous, intraarterial, intraocular, intranasal, intramuscular, and subcutaneous administration. The chemotherapeutic agent may be administered to cancer patients once a day, twice a day, three times a day, once a week, twice a week, three times a week, four times a week, five times a week, six times a week, seven times a week, bi-weekly, tri-weekly, monthly, every two months, every three months, every four months, every five months, twice a year, three times a year, four times a year, five times a year, or six times a year. The specific dosage and administration schedule for a chemotherapeutic agent may be determined by a skilled physician based on a number of factors including the age, weight, and sex of the patient, the type of cancer, and the severity of one or more symptoms of cancer.

In instances where a cancer patient is administered the combination of one or more (e.g., two, three, four, five, or six) PARN inhibitors and one or more (e.g., two, three, four, five, or six) chemotherapeutic agents, the one or more PARN inhibitors and the one or more chemotherapeutic agents may be administered at the same time (e.g., administered in the same formulated dose). In another example, the one or more PARN inhibitors may be administered to the cancer patient prior to the administration of the one or more chemotherapeutic agents (e.g., wherein the bioactive period of the one or more PARN inhibitors overlaps with the bioactive period of the one or more chemotherapeutic agents).

In a further example, the one or more chemotherapeutic agents may be administered to the cancer patient prior to the administration of the one or more PARN inhibitors (e.g., wherein the bioactive period of the one or more PARN inhibitors overlaps with the bioactive period of the one or more chemotherapeutic agents). The therapeutic methods provided by this disclosure may be performed alone or in conjunction with another cancer therapy and may be provided at home, the doctor's office, a clinic, a hospital's outpatient department, or a hospital. Treatment generally begins at a hospital so that the doctor can observe the therapy's effects closely and make any adjustments that are needed. The duration of the therapy depends on the age and condition of the patient, the stage of the patient's cancer, and how the patient responds to the treatment. Additionally, a person having a greater risk of developing cancer may be treated by the methods of this disclosure (e.g., a person who is genetically predisposed). Therapy, as provided by this disclosure, may be given in on-and-off cycles that include rest periods so that the patient's body has a chance to build healthy new cells and regain its strength. Therapy may be used to extend the patient's lifespan.

For cancer treatment, depending on the type of cancer and its stage of development, the therapy can be used to slow the spread of the cancer, to slow the cancer's growth, to kill or arrest cancer cells that may have spread to other parts of the body from the original tumor, or to relieve symptoms caused by the cancer.

### Combination Therapies

In addition to the PARN inhibitors, chemotherapeutic agents, or the combination of PARN inhibitors and chemotherapeutic agents described above, the cancer patient may also be treated with one or more (e.g., two, three, four, or five) additional agents including one or more (e.g., one, two, three, four, or five) non-steroidal anti-inflammatory drug(s) (NSAID(s)), one or more (e.g., two, three, four, or five) immunosuppressive agent(s), one or more (e.g., two, three, four, or five) calcineurin inhibitor(s), or one or more (e.g., two, three, four, or five) analgesic(s). Examples of NSAIDs, immunosuppressive agents, and analgesics are known in the art.

Depending on the type of cancer and its stage of development, the combination therapy can be used to treat cancer, to slow the spreading of the cancer, to slow the cancer's growth, to kill or arrest cancer cells that may have spread to other parts of the body from the original tumor, to relieve symptoms caused by the cancer, or to prevent cancer in the first place. Combination therapy can also help people live more comfortably by eliminating cancer cells that cause pain or discomfort.

The administration of any of the above combinations of agents (e.g., combination of PARN inhibitors and chemotherapeutic agents) of the present invention allows for the administration of lower doses of each compound, providing similar efficacy and lower toxicity compared to administration of either compound alone. Alternatively, such combinations result in improved efficacy in treating cancer with similar or reduced toxicity.

The methods provided by this disclosure may be used to treat an individual having any type of cancer (e.g., an individual diagnosed as having a cancer). Non-limiting examples of cancer that may be treated by the provided methods include: acoustic neuroma, acute leukemia, acute lymphocytic leukemia, acute monocytic leukemia, acute myeloblastic leukemia, acute myelocytic leukemia, acute myelomonocytic leukemia, acute promyelocytic leukemia, acute erythroleukemia, adenocarcinoma, angiosarcoma, astrocytoma, basal cell carcinoma, bile duct carcinoma, bladder carcinoma, brain cancer, breast cancer, bronchogenic carcinoma, cervical cancer, chondrosarcoma, chordoma, choriocarcinoma, chronic leukemia, chronic lymphocytic leukemia, chronic myelocytic leukemia, colon cancer, colon carcinoma, craniopharyngioma, cystadenocarcinoma, embryonal carcinoma, endotheliosarcoma, ependymoma, epithelial carcinoma, Ewing's tumor, glioma, heavy chain disease, hemangioblastoma, hepatoma, Hodgkin's disease, large cell carcinoma, leiomyosarcoma, liposarcoma, lung cancer, lung carcinoma, lymphangioendotheliosarcoma, lymphangiosarcoma, macroglobulinemia, medullary carcinoma, medulloblastoma, melanoma, meningioma, mesothelioma, myxosarcoma, neuroblastoma, non-Hodgkin's disease, oligodendroglioma, osteogenic sarcoma, ovarian cancer, pancreatic cancer, papillary adenocarcinomas, papillary carcinoma, pincaloma, polycythemia vera, prostate cancer, rhabdomyosarcoma, renal cell carcinoma, retinoblastoma, schwannoma, sebaceous gland carcinoma, seminoma, small cell lung carcinoma, squamous cell carcinoma, sweat gland carcinoma, synovioma, testicular cancer, uterine cancer, Waldenstrom's fibrosarcoma, and Wilm's tumor.

A skilled physician may monitor the effectiveness of treatment of a cancer by monitoring the severity or duration of one or more symptoms of cancer. Non-limiting examples of symptoms of cancer include: blood in urine, pain or burning upon urination, cloudy urine, pain in bone, fractures in bones, fatigue, weight loss, repeated infections, nausca, vomiting, constipation, numbness in the legs, bruising, dizziness, drowsiness, abnormal eye movements, changes in vision, changes in speech, headaches, thickening of a tissue, rectal bleeding, abdominal cramps, loss of appetite, fever, enlarged lymphnodes, persistent cough, blood in sputum, lung congestion, itchy skin, lumps in skin, abdominal swelling, vaginal bleeding, jaundice, heartburn, indigestion, cell proliferation, and loss of regulation of controlled cell death.

The methods of treatment provided by this disclosure may result in at least a 5% (e.g., at least 10%, 15%, 20%, 25%, 30%, 35%, 40%, 45%, 50%, 55%, 60%, 65%, 70%, 75%, 80%, 85%, 90%, or 95% decrease) in one or more symptoms (e.g., two, three, four, or five symptoms) of cancer (e.g., those symptoms listed above). The methods of treatment may also provide a decrease in the toxicity normally observed for a PARN inhibitor and/or a chemotherapeutic agent. The methods of treatment may also provide for a reduction in the dosage of a PARN inhibitor or a chemotherapeutic agent necessary to achieve a therapeutic effect (e.g., a reduction in one or more symptoms of cancer). Desirably, the provided methods may result in a decrease in the metastasis or recurrence of cancer in a patient or may provide for an increase in the duration of remission in a patient.

Patients with cancer cell(s) that have an active p53 signaling pathway are more sensitive to chemotherapeutic agents (e.g., DNA damaging agents). Thus, in determining the treatment regime for a cancer patient, a physician may suggest the administration of one or more chemotherapeutic agent(s) (e.g., an additional dosage of a chemotherapeutic agent) to a patient having cancer cell(s) with an inactivated p53 signaling pathway (e.g., a patient having cancer cell(s) with active or elevated PARN and an inactivated p53 signaling pathway). Similarly, such patient may selectively benefit from the administration of one or more PARN inhibitor(s) or a combination of one or more PARN inhibitor(s) and one or more chemotherapeutic agent(s). Thus, this disclosure provides methods that allow a physician to identify a specific subset of patients that may selectively benefit from the administration of one or more chemotherapeutic agent(s), or administration of one or more PARN inhibitor(s) or the combination of one or more PARN inhibitor(s) and one or more chemotherapeutic agent(s) (e.g., cancer patients having cancer cell(s) with active or elevated PARN and, optionally, an inactivated p53 pathway). These methods require steps for the determination of the activity or expression level of PARN and, optionally, steps for the determination of the inactivation of the p53 pathway. These methods allow a physician to identify a patient that may selectively benefit from the administration of a PARN inhibitor, a chemotherapeutic agent, or a combination of a PARN inhibitor and a chemotherapeutic agent. The identified patient would experience at least a 10% decrease in one or more symptoms of cancer relative to another cancer patient receiving the same treatment.

This disclosure further provides methods for diagnosing a chemotherapy-sensitive or a chemotherapy-resistant cancer in a patient. As described above, cancer cells having an inactivated p53 pathway (e.g., cancer cells having inactive or repressed expression of PARN and an active p53 pathway) are more sensitive to treatment with one or more chemotherapeutic agent(s) (e.g., an agent that induces genotoxic stress, such as an agent that induces DNA damage) compared to non-cancer cells or other cancer cells (e.g., cells having active or elevated PARN and an inactive p53 pathway). In addition, such cancer cells are more sensitive to treatment with one or more PARN inhibitor(s) or a combination of one or more PARN inhibitor(s) and one or more chemotherapeutic agent(s) compared to non-cancer cells or other cancer cells having an active p53 pathway. Thus, these methods allow for the diagnosis of a chemotherapy-sensitive cancer in patient by measuring the activity or expression of PARN and, optionally, measuring the activity or expression of the p53 pathway in a cancer cell from the patient, wherein a patient having cancer cell(s) with inactivate or repressed p53 are diagnosed as having a chemotherapy-resistant cancer (e.g., indicating that these patients have cancer that may be insensitive to treatment that includes the administration of one or more chemotherapeutic agents). This disclosure also provides methods for diagnosing of a chemotherapy-sensitive cancer in a patient by measuring the activity or expression of PARN and, optionally, measuring the activity or expression of the p53 pathway in a cancer cell from the patient, wherein a patient having cancer cell(s) with inactivated or repressed PARN are diagnosed as having a chemotherapy-sensitive cancer (e.g., indicating that these patients have cancer that may be resistant to treatment that includes the administration of one or more PARN inhibitors).

This disclosure further provides kits that provide reagents for diagnosing a chemotherapy-resistant cancer or a chemotherapy-sensitive cancer in a subject. For example, such kits may contain one or more reagent(s) (e.g., two, three, four, five, or six reagents) capable of measuring one or more feature(s) (e.g., two, three, four, five, or six features) in a cancer cell(s) from a patient selected from the group of: cytoplasmic or nuclear PARN protein localization, phosphorylation of total PARN protein, levels of phosphorylated PARN protein in the cytoplasm or nucleus, and one or more reagents (e.g., two, three, four, five, or six reagents) capable of capable of measuring one or more feature(s) (e.g., two, three, four, five, or six features) in a cancer cell(s) from said patient selected from the group consisting of: tumor protein-53 (p53) mRNA or protein levels, expression of a mutant or truncated p53 with decreased expression or activity, and p21 expression or activity. The kits may further include instructions for using the above reagents to determine the presence of a chemotherapy-resistant or chemotherapy-sensitive cancer in the patient.

Non-limiting examples of reagents that may be provided in the kits include: antibodies that bind to phosphorylated, nonphosphorylated, or total PARN protein; antibodies that bind to p53; an oligonucleotide containing a sequence complementary to a nucleic acid sequence encoding p53 (e.g., encoding wild type p53 protein or a mutant or truncated p53 protein); nucleic acid primers that may be used to amplify a p53 mRNA or gene (e.g., a mRNA or gene encoding wild type p53 protein or a mRNA or gene encoding mutant or truncated p53 protein).

The instructions provided with the kit may describe that the use of one or more of the above reagents to measure one or more (e.g., two, three, four, or five) features of PARN pathway activation or one or more (e.g., two, three, four, or five) features of PARN activity or expression, and, optionally, the use of one of more of the above reagents to measure one or more (e.g., two, three, four, or five) features of p53 pathway inactivation.

Using the reagents provided in the kits, PARN activity or expression may be indicated by the observance of one or more (e.g., two, three, four, five, or six) of following features: increased (e.g., by at least 10%, 20%, 30%, 40%, 50%, 60%, 70%, 80%, or 90%) PARN protein in the cytoplasm, decreased (e.g., by at least 10%, 20%, 30%, 40%, 50%, 60%, 70%, 80%, or 90%) PARN in the nucleus, increased (e.g., by at least 10%, 20%, 30%, 40%, 50%, 60%, 70%, 80%, or 90%) total PARN protein phosphorylation, increased (e.g., by at least 10%, 20%, 30%, 40%, 50%, 60%, 70%, 80%, or 90%) levels of phosphorylated PARN in the cytoplasm or nucleus.

Conversely, inactivated p53 signaling pathway may be indicated by the observance of one or more (e.g., two, three, four, five, or six) of the following features: decreased (by at least 10%, 20%, 30%, 40%, 50%, 60%, 70%, 80%, or 90%) p53 protein in the cytoplasm, increased (by at least 10%, 20%, 30%, 40%, 50%, 60%, 70%, 80%, or 90%) PARN in the nucleus, decreased (by at least 10%, 20%, 30%, 40%, 50%, 60%, 70%, 80%, or 90%) total PARN protein phosphorylation, decreased (by at least 10%, 20%, 30%, 40%, 50%, 60%, 70%, 80%, or 90%) levels of phosphorylated PARN in the cytoplasm or nucleus.

p53 pathway inactivation is indicated by the observance of one or more (e.g., two, three, four, five, or six) of the following features: decreased (e.g., by at least 10%, 20%, 30%, 40%, 50%, 60%, 70%, 80%, or 90%) p53 mRNA or protein levels, expression of a mutant or truncated p53 with decreased (e.g., by at least 10%, 20%, 30%, 40%, 50%, 60%, 70%, 80%, or 90%) expression or activity, and decreased (by at least 10%, 20%, 30%, 40%, 50%, 60%, 70%, 80%, or 90%) p21 expression or activity.

The above features of PARN activity and p53 pathway inactivation may be performed using a sample of cells from a patient (e.g., a biopsy sample or blood sample) or a cellular lysate prepared from cells from a patient. A patient that is measured as having cells with one or more features of PARN activity and, optionally, one or more features of p53 pathway activity, is diagnosed as having a chemotherapy-resistant cancer (e.g., a patient that may benefit from the administration of one or more PARN inhibitor(s) or the combination of one or more PARN inhibitor(s) and one or more chemotherapeutic agent(s)). A patient that is measured as having cells with one or more features of PARN inactivity and, optionally, one or more features of p53 pathway activity, is diagnosed as having a chemotherapy-sensitive cancer (e.g., a patient that may benefit from administration of one or more chemotherapeutic agent(s)). Compositions Identified by Screening

PARN protein and/or RNA can be used as targets for any combinatorial technique to identify molecules or macromolecular molecules that interact with PARN in a desired way. The nucleic acids, peptides, proteins and related molecules disclosed herein can be used as targets for the combinatorial approaches. Also disclosed are the compositions that are identified through such screening/combinatorial techniques in which PARN protein and/or RNA are used as the target in a combinatorial or screening protocol.

When using the PARN protein and/or RNA in combinatorial techniques or screening methods, molecules, such as macromolecular molecules, will be identified that have particular desired properties such as inhibition or stimulation of the PARN molecule's function.

Combinatorial chemistry includes but is not limited to all methods for isolating small molecules or macromolecules that are capable of binding either a small molecule or another macromolecule, typically in an iterative process. Proteins, oligonucleotides, and sugars are examples of such macromolecules.

There are a number of methods for isolating proteins which either have de novo activity or a modified activity. For example, phage display libraries have been used to isolate numerous peptides that interact with a specific target. (See for example, U.S. Pat. Nos. 6,031,071; 5,824,520; 5,596,079; and 5,565,332 which are herein incorporated by reference at least for their material related to phage display and methods relate to combinatorial chemistry)

A preferred method for isolating proteins that have a given function is described by Roberts and Szostak (Roberts R. W. and Szostak J. W. Proc. Natl. Acad. Sci. USA, 94 (23) 12997-302 (1997). This combinatorial chemistry method couples the functional power of proteins and the genetic power of nucleic acids. An RNA molecule is generated in which a puromycin molecule is covalently attached to the 3′-end of the RNA molecule. An in vitro translation of this modified RNA molecule causes the correct protein, encoded by the RNA to be translated. In addition, because of the attachment of the puromycin, a peptdyl acceptor which cannot be extended, the growing peptide chain is attached to the puromycin which is attached to the RNA. Thus, the protein molecule is attached to the genetic material that encodes it. Normal in vitro selection procedures can now be done to isolate functional peptides. Once the selection procedure for peptide function is complete traditional nucleic acid manipulation procedures are performed to amplify the nucleic acid that codes for the selected functional peptides. After amplification of the genetic material, new RNA is transcribed with puromycin at the 3′-end, new peptide is translated and another functional round of selection is performed. Thus, protein selection can be performed in an iterative manner just like nucleic acid selection techniques. The peptide which is translated is controlled by the sequence of the RNA attached-to the puromycin. This sequence can be anything from a random sequence engineered for optimum translation (i.e. no stop codons etc.) or it can be a degenerate sequence of a known RNA molecule to look for improved or altered function of a known peptide. The conditions for nucleic acid amplification and in vitro translation are well known to those of ordinary skill in the art and are preferably performed as in Roberts and Szostak (Roberts R. W. and Szostak J. W. Proc. Natl. Acad. Sci. USA, 94 (23) 12997-302 (1997)).

Another preferred method for combinatorial methods designed to isolate peptides is described in Cohen et al. (Cohen B. A., et al., Proc. Natl. Acad. Sci. USA 95 (24): 14272-7 (1998)). This method utilizes and modifies two-hybrid technology. Yeast two-hybrid systems are useful for the detection and analysis of protein: protein interactions. The two-hybrid system, initially described in the yeast Saccharomyces cerevisiae, is a powerful molecular genetic technique for identifying new regulatory molecules, specific to the protein of interest (Fields and Song, Nature 340:245-6 (1989)). Cohen et al., modified this technology so that novel interactions between synthetic or engineered peptide sequences could be identified which bind a molecule of choice.

Using methodology well known to those of skill in the art, in combination with various combinatorial libraries, one can isolate and characterize those small molecules or macromolecules, which bind to or interact with PARN. The relative binding affinity of these compounds can be compared and optimum compounds identified using competitive binding studies, which are well known to those of skill in the art.

Techniques for making combinatorial libraries and screening combinatorial libraries to isolate molecules that bind a desired target are well known to those of skill in the art. Representative techniques and methods can be found in but are not limited to U.S. Pat. Nos. 5,084,824, 5,288,514, 5,449,754, 5,506,337, 5,539,083, 5,545,568, 5,556,762, 5,565,324, 5,565,332, 5,573,905, 5,618,825, 5,619,680, 5,627,210, 5,646,285, 5,663,046, 5,670,326, 5,677,195, 5,683,899, 5,688,696, 5,688,997, 5,698,685, 5,712,146, 5,721,099, 5,723,598, 5,741,713, 5,792,431, 5,807,683, 5,807,754, 5,821,130, 5,831,014, 5,834,195, 5,834,318, 5,834,588, 5,840,500, 5,847,150, 5,856,107, 5,856,496, 5,859,190, 5,864,010, 5,874,443, 5,877,214, 5,880,972, 5,886,126, 5,886,127, 5,891,737, 5,916,899, 5,919,955, 5,925,527, 5,939,268, 5,942,387, 5,945,070, 5,948,696, 5,958,702, 5,958,792, 5,962,337, 5,965,719, 5,972,719, 5,976,894, 5,980,704, 5,985,356, 5,999,086, 6,001,579, 6,004,617, 6,008,321, 6,017,768, 6,025,371, 6,030,917, 6,040,193, 6,045,671, 6,045,755, 6,060,596, and 6,061,636.

Combinatorial libraries can be made from a wide array of molecules using a number of different synthetic techniques. For example, libraries containing fused 2,4-pyrimidinediones (U.S. Pat. No. 6,025,371) dihydrobenzopyrans (U.S. Pat. Nos. 6,017,768 and 5,821,130), amide alcohols (U.S. Pat. No. 5,976,894), hydroxy-amino acid amides (U.S. Pat. No. 5,972,719) carbohydrates (U.S. Pat. No. 5,965,719), 1,4-benzodiazepin-2,5-diones (U.S. Pat. No. 5,962,337), cyclics (U.S. Pat. No. 5,958,792), biaryl amino acid amides (U.S. Pat. No. 5,948,696), thiophenes (U.S. Pat. No. 5,942,387), tricyclic Tetrahydroquinolines (U.S. Pat. No. 5,925,527), benzofurans (U.S. Pat. No. 5,919,955), isoquinolines (U.S. Pat. No. 5,916,899), hydantoin and thiohydantoin (U.S. Pat. No. 5,859,190), indoles (U.S. Pat. No. 5,856,496), imidazol-pyrido-indole and imidazol-pyrido-benzothiophenes (U.S. Pat. No. 5,856,107) substituted 2-methylene-2,3-dihydrothiazoles (U.S. Pat. No. 5,847,150), quinolines (U.S. Pat. No. 5,840,500), PNA (U.S. Pat. No. 5,831,014), containing tags (U.S. Pat. No. 5,721,099), polyketides (U.S. Pat. No. 5,712,146), morpholino-subunits U.S. Pat. Nos. 5,698,685 and 5,506,337), sulfamides (U.S. Pat. No. 5,618,825), and benzodiazepines (U.S. Pat. No. 5,288,514).

Screening molecules for inhibition of PARN is a method of isolating desired compounds. In one embodiment, the inhibitors are non-competitive inhibitors PARN. One type of non-competitive inhibitor will cause allosteric rearrangements which prevent binding of PARN to RNA.

As used herein combinatorial methods and libraries include traditional screening methods and libraries as well as methods and libraries used in interative processes.

The disclosed compositions can be used as targets for any molecular modeling technique to identify either the structure of PARN inhibitors or to identify potential or actual molecules, such as small molecules, which interact in a desired way with PARN. The nucleic acids, peptides, and related molecules disclosed herein can be used as targets in any molecular modeling program or approach.

When using the disclosed compositions in modeling techniques, molecules, such as macromolecular molecules, will be identified that have particular desired properties such as inhibition or stimulation of PARN function. Thus, the products produced using the molecular modeling approaches that involve PARN are included within the compounds of this disclosure.

Thus, one way to isolate molecules that bind a molecule of choice is through rational design. This is achieved through structural information and computer modeling. Computer modeling technology allows visualization of the three-dimensional atomic structure of a selected molecule and the rational design of new compounds that will interact with the molecule. The three-dimensional construct typically depends on data from x-ray crystallographic analyses or NMR imaging of the selected molecule. The molecular dynamics require force field data. The computer graphics systems enable prediction of how a new compound will link to the target molecule and allow experimental manipulation of the structures of the compound and target molecule to perfect binding specificity. Prediction of what the molecule-compound interaction will be when small changes are made in one or both requires molecular mechanics software and computationally intensive computers, usually coupled with user-friendly, menu-driven interfaces between the molecular design program and the user.

Examples of molecular modeling systems are the CHARMm and QUANTA programs, Polygen Corporation, Waltham, Mass. CHARMm performs the energy minimization and molecular dynamics functions. QUANTA performs the construction, graphic modeling and analysis of molecular structure. QUANTA allows interactive construction, modification, visualization, and analysis of the behavior of molecules with each other.

A number of articles review computer modeling of drugs interactive with specific proteins, such as Rotivinen, et al., 1988 Acta Pharmaceutica Fennica 97, 159-166; Ripka, New Scientist 54-57 (Jun. 16, 1988); McKinaly and Rossmann, 1989 Annu. Rev. Pharmacol. Toxiciol. 29, 111-122; Perry and Davies, QSAR: Quantitative Structure-Activity Relationships in Drug Design pp. 189-193 (Alan R. Liss, Inc. 1989); Lewis and Dean, 1989 Proc. R. Soc. Lond. 236, 125-140 and 141-162; and, with respect to a model enzyme for nucleic acid components, Askew, et al., 1989 J. Am. Chem. Soc. 111, 1082-1090. Other computer programs that screen and graphically depict chemicals are available from companies such as BioDesign, Inc., Pasadena, Calif., Allelix, Inc, Mississauga, Ontario, Canada, and Hypercube, Inc., Cambridge, Ontario. Although these are primarily designed for application to drugs specific to particular proteins, they can be adapted to design of molecules specifically interacting with specific regions of DNA or RNA, once that region is identified.

Although described above with reference to design and generation of compounds which could alter binding, one could also screen libraries of known compounds, including natural products or synthetic chemicals, and biologically active materials, including proteins, for compounds which alter substrate binding or enzymatic activity.

These compositions comprising PARN protein and/or RNA may be used as targets in combinatorial chemistry protocols or other screening protocols to isolate molecules that possess desired functional properties related to modulation of PARN activity and/or p53 activity.

The disclosed compositions can also be used as diagnostic tools related to cancers listed above that result from aberrant deadenylation or p53 activity.

PARN compositions can be used as discussed herein as either reagents in microarrays or as reagents to probe or analyze existing microarrays. PARN compositions can also be used in any known method of screening assays, related to chip/microarrays. PARN compositions can also be used in any known way of using the computer readable embodiments of the disclosed compositions, for example, to study relatedness or to perform molecular modeling analysis related to the disclosed compositions.

Preferred embodiments include methods of screening for a substance that modulates PARN activity comprising incubating the substance with a PARN protein or RNA, and assaying for a change in PARN activity and/or p53 activity and/or p53 signaling, and/or adenylation/deadenylation activity, indicating a PARN modulating substance. Such a modulator preferably modulates carcinogenesis, cancer progression, and/or metastasis.

These screening methods may include incubating a test substance with a stably transfected cell comprising a reporter gene and assaying the amount of PARN activity and/or p53 activity and/or p53 signaling and/or adenylation/deadenylation activity in the cell. For example, an increase or decrease in the amount of PARN mRNA relative to the amount of mRNA in the absence of the substance indicates a substance that modulates PARN activity.

These screening methods may include screening for a substance that modulates p53 signaling including administering a substance to a screening system wherein the system comprises the components essential for p53 signaling, assaying the effect of the substance on the amount of p53 activity in the system, wherein a substance which causes a change in the amount of p53 activity present in the system compared to the amount of p53 activity in the system in the absence of composition is a modulator of PARN.

Each publication or patent cited herein is incorporated herein by reference in its entirety.

The features and other details of this disclosure will now be more particularly described and pointed out in the following examples describing preferred techniques and experimental results. These examples are provided for the purpose of illustrating this disclosure and should not be construed as limiting.

### EXAMPLES

The following methods and materials were used in conducting the experimental Examples described below.

Cell culture: HeLa cells were purchased from ATCC and verified for mycoplasma contamination. Hek293T wild-type and Dicer knockout cells were a kind gift from Prof. Christopher Sullivan at University of Texas at Austin. Cells were cultured in DMEM containing 10% FBS, 1% Pen/Strep, 1× Glutamax and Normocin at 37° C. under ambient conditions. Cells were passaged every three days and sub-cultured upon reaching 80% confluency.

RNA interference in HeLa cells: HeLa cells were seeded approx. 100,000 cells/well in a six-well plate 24 hours before transfection. siRNA transfection was performed using Interferin (Polyplus) to a final concentration of 5 nM per well as per manufacturer's protocol. 72 hours after transfection, cells were collected for either RNA or protein analysis.

PARN plasmid co-transfection in HeLa cells: siRNA transfection of PARN siRNA was repeated as above. 24 hours after siRNA transfection, 1 μg of a GFP or PARN plasmid was transfected using JetPrime (Polyplus) as per manufacturer's protocol. Cells were harvested for protein analysis 48 hours after plasmid transfection.

siRNAs and plasmid: siRNAs targeting PARN (siGenome), PAPD5 (On-Target plus), DIS3L (On-Target plus) and DIS3L2 (On-Target plus) were purchased from Dharmacon in the Smartpool formulation. All-stars negative control siRNA from Qiagen was used as negative control. PARN plasmid was a gift from Prof. Yukihide Tomari at University of Tokyo, Japan.

RNA extraction and northern blotting: Total RNA was extracted from cell lysates using Quick RNA mini-prep kit from Zymo Research as per manufacturer's protocol. After quantification on Nanodrop, 15 μg of total RNA was separated on a 10% acrylamide 7M Urea gel. RNA was transferred to a nylon membrane (Nytran SPC, GE Healthcare) using wet transfer at 4° C. After UV crosslinking, the blot was pre-hybridized and hybridized in PerfectHyb Plus Hybridization Buffer (Sigma Aldrich) at 42° C. miRNA LNA probes for each target miRNA were purchased from Exiqon. Probe against 5s rRNA has been described previously (Shukla, S., et al., Nature Structural & Molecular Biology 23:286-92 (2016)). After hybridization and washing in 2×SSC 0.1% SDS wash buffer, blots were exposed to a cassette and imaged on a Typhoon FLA 9500 Phosphoimager. Band intensities were quantified using ImageQuant TL.

Western blotting: 10 μg of total protein was separated on a 4%-12% Bis-Tris NuPage gel (ThermoFisher), and transferred to a protran membrane (Amersham). Antibodies against p53 used in this study were the 7F5 clone (Cell Signaling) and DO-1 (Santa Cruz). Antibodies against PARN and Gapdh have been described previously (Shukla, S., et al., Nature Structural & Molecular Biology (2016), supra).

DNA damage and imaging: Doxorubicin and Etoposide were purchased from Sigma Aldrich. For DNA damage treatment, Dox or EP was added to a final concentration of luM or 10 μM respectively, 48 hours after siRNA transfection. Chemical treatment was allowed to take place for 24 hours, after which cells were either harvested for protein analysis, or imaged on an EVOS FL cell imaging system. For cell viability measurement, equal number of cells were stained with trypan blue, and viable cells were counted on a hemocytometer.

Small RNA sequencing: 1 μg of total RNA was used as input for library preparation using the NEXTflex Small RNA Library prep kit V3 for Illumina from Bioo Scientific. Libraries were sequenced on an Illumina Next Seq sequencer using the 1×150 cycle kit. Approximately 8 million unpaired reads were obtained for each library. After quality filtering and adapter trimming, including trimming of 4N bases from the 5′ and 3′ ends of reads, reads were mapped to the mature miRNA 21 database from miRbase using the blastn tool from NCBI Blast software. Best matches were selected according to the lowest q value, and counted using a custom python script. Abundance of miRNAs was calculated and normalized internally to reads per million. miRNA reads were plotted on R using the ggplot2 library.

3′ end sequencing of miRNAs: 3′ end sequencing was performed using a previously described protocol (Goldfarb, K. C. and Cech, T. R. BMC Mol. Biol. 14:23 (2013)). Reverse primer for 3′ RACE was selected as the first 20 bases of the mature miR-21-5p or miR-181b-5p. Libraries were sequenced on an Illumina Next Seq sequencer using the 1x 150 cycle kit. Approximately 3 million reads were obtained for each library. Reads of interest were selected using the search sequence corresponding to the miRNA and the 3′ appendix. Canonical 3′ end for miR-21-5p was defined as described previously (Boele, J. et al. Proceedings of the National Academy of Sciences 111:11467-72 (2014)). Canonical 3′ end for miR-181b-5p was defined as listed on miRbase. Statistics were performed using two-proportion Z-score test, and Z scores were converted to P values to identify statistically significant differences.

### Example 1: PARN Regulates the Stability of a Diverse miRNAs in Human Cells

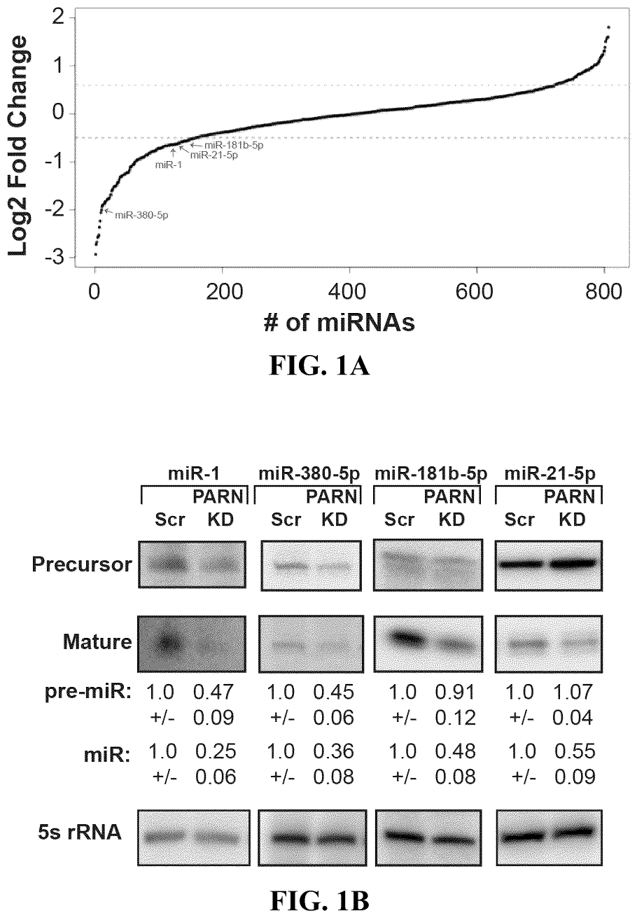

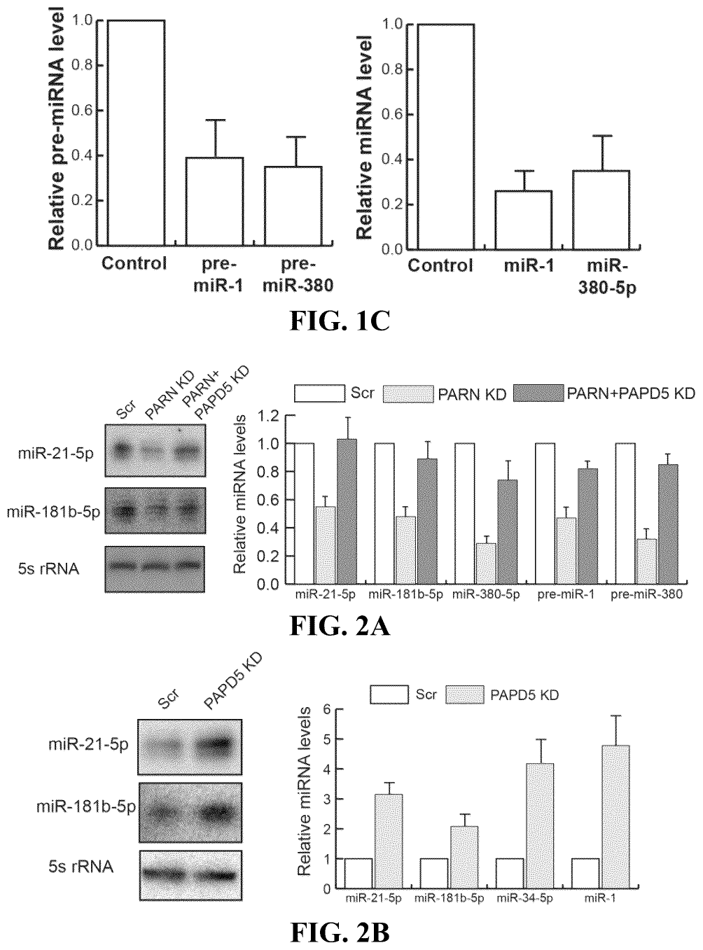

miRNAs are small non-coding RNAs (ncRNAs) that regulate gene expression through their ability to base pair with complementary regions in target mRNAs, by sequencing miRNA populations from control and PARN knockdown Hela cells. From four biological replicates, we identified 86 miRNAs that were upregulated more than 1.5-fold in PARN knockdown cells, and 157 miRNAs that were downregulated more than 0.7-fold in PARN knockdown cells (FIG. 1A) demonstrating that PARN affects multiple miRNAs in the cell.

To verify our sequencing results, and to determine if PARN knockdown was reducing mature miRNAs or pre-miRNAs, we examined the levels of mature and precursor miRNAs by other methods. We found that miR-1 and miR-380-5p are reduced at the pre-miRNA form using northern blots and RT-qPCR, with the levels of the mature miRNA similar to the pre-miRNA upon PARN knockdown (FIGS. 1B and 1C). This suggest the reduction in these miRNAs occurs at the precursor stage. In contrast, miR-181b-5p or miR-21-5p were not affected at precursor level, and only the mature forms were reduced upon PARN knockdown (FIG. 1B). These observations validate our sequencing results and argue that PARN can affect the levels of either the precursor or mature forms of miRNAs in a miRNA-specific manner.

A previous study showed that PARN knockdown led to an increase in an adenylated form of miR-21-5p in human cells (Boele, J. et al. Proceedings of the National Academy of Sciences 111:11467-72 (2014)), suggesting that PARN degrades miR-21-5p. In direct contrast, we observed a reduction in miR-21-5p upon PARN knockdown in our sequencing data, which we verified by northern blots (FIG. 1B), suggesting that PARN actually stabilizes miR-21-5p by deadenylating an adenylated intermediate which would otherwise be degraded by 3′ to 5′ exonucleases.

### Example 2: PAPD5 Destabilizes miRNAs in the Absence of PARN

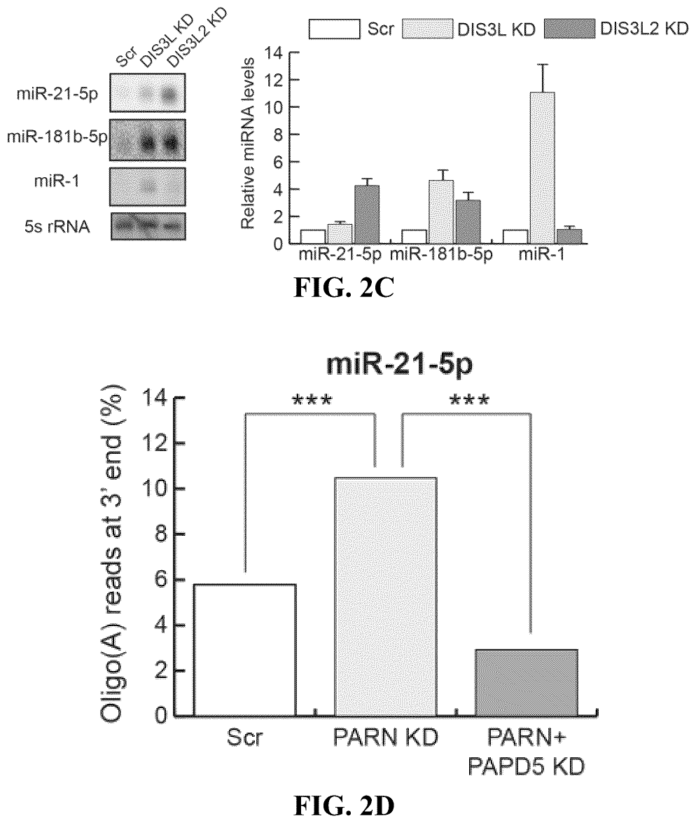

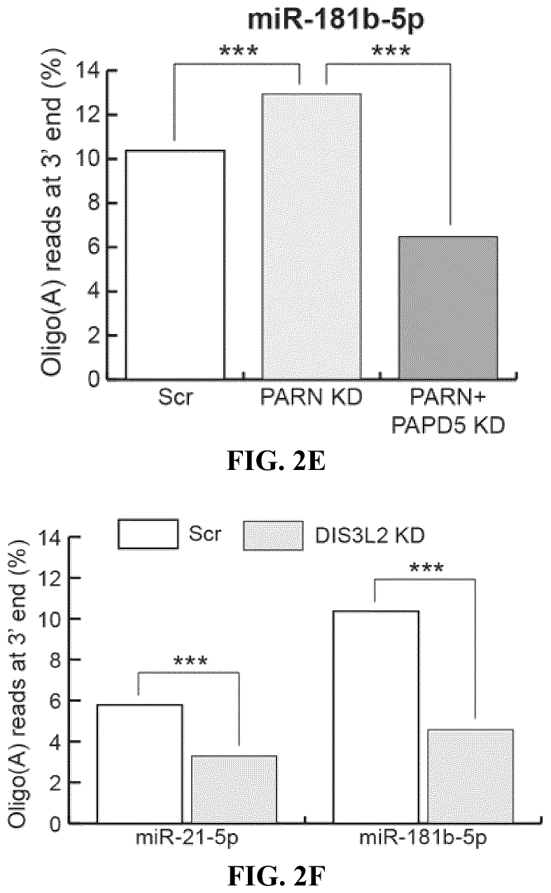

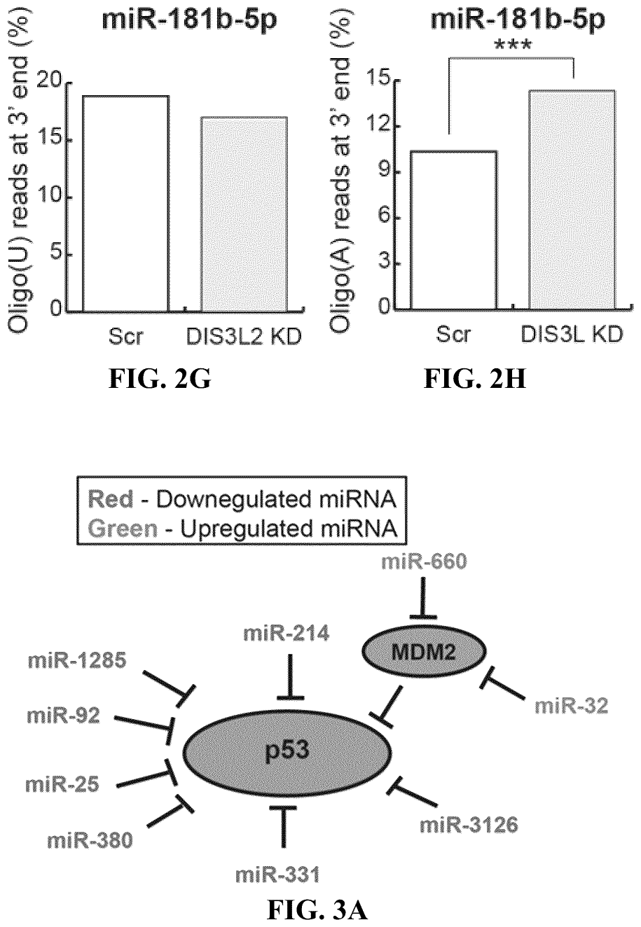

Previous studies on PARN-mediated stabilization of other ncRNAs suggests that PARN removes oligo (A) tails added by PAPD5 from the 3′ end of its substrates, which would otherwise lead to the degradation of the ncRNA by a competing 3′ to 5′ exonuclease recruited by the oligo (A) tail. We therefore investigated whether PAPD5 co-knockdown rescues the reduction in miRNA levels upon PARN knockdown. We found that PAPD5 co-knockdown was sufficient to rescue the reduced levels of pre-miR-380 and pre-miR-1 in PARN knockdown (FIG. 2A). Similarly, PAPD5 and PARN co-knockdown rescued the levels of mature miR-21-5p, miR-181b-5p and miR-380-5p (FIG. 2A), suggesting that depletion of the adenylating enzyme protects pre- or mature miRNAs from degradation by a 3′ to 5′ exonuclease. This is in contrast to previous studies where adenylation of miRNA 3′ end was suggested to stabilize miRNAs and protect them from degradation. Thus, PARN may function to remove oligo (A) tails on miRNAs, limiting miRNA decay by competing 3′ to 5′ exonucleases.

To determine if adenylation and 3′ to 5′ exonuclease degradation played an important role in modulating miRNA levels in the presence of PARN, we investigated whether PAPD5 knockdown alone affected miRNA levels in human cells. We found that PAPD5 knockdown by itself led to an average 3-fold increase in the levels of all miRNAs we tested, including miR-181b-5p, miR-1, miR-34a-5p, and miR-21-5p (FIG. 2B). Importantly, PAPD5 knockdown alone did not affect the levels of the precursor miRNAs, suggesting that the mature miRNA is more sensitive to 3′ end adenylation in the presence of PARN. Together, these data show that PAPD5-mediated adenylation is a global mechanism for regulating miRNA levels in human cells by recruiting 3′ to 5′ exonucleases leading to miRNA degradation.

### Example 3: DIS3L and DIS3L2 are Critical 3′ To′5′ Exonucleases for Regulating miRNA Stability in Human Cells

To identify the enzymes that degrade adenylated miRNAs, we focused on the two predominant cytoplasmic 3′ to 5′ exonucleases, DIS3L and DIS3L2, which degrade a variety of ncRNA substrates in human cells. DIS3L2 has also been shown to degrade uridylated pre-miRNA in human cells, although whether it can also target adenylated miRNAs has not been examined. We found that DIS3L knockdown led to an eleven-fold increase in the levels of miR-1, a four-fold increase in the levels of miR-181b-5p but had only minor effects on miR-380-5p (1.1×) or miR-21-5p (1.4×) (FIG. 2C). In contrast, DIS3L2 knockdown increased miR-21-5p (4X), miR-181b-5p (3X), miR-380-5p (4.5X) but had no effect on miR-1 (FIG. 2C). This demonstrates that both DIS3L and DIS3L2 modulate miRNA levels in a miRNA-specific manner.

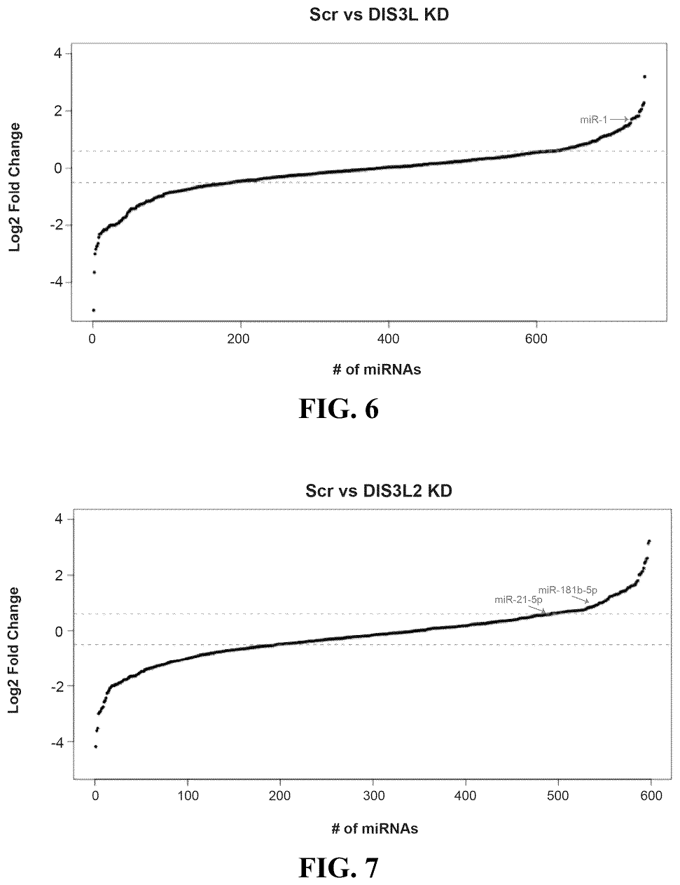

To investigate whether DIS3L or DIS3L2 regulate the stability of miRNAs globally in human cells, we sequenced miRNA libraries from DIS3L and DIS3L2 knockdown cells and found that DIS3L knockdown led to global changes in miRNA levels; out of 746 miRNAs, 129 miRNAs were upregulated more than 1.5-fold in DIS3L knockdown cells, and 185 miRNAs were downregulated more than 0.7-fold in DIS3L knockdown cells compared to control cells (FIG. 6). Similarly, DIS3L2 knockdown led to global changes in miRNA population in Hela cells; out of 598 miRNAs, 106 miRNAs were upregulated more than 1.5-fold, and 194 miRNAs were downregulated more than 0.7-fold in DIS3L2 knockdown cells (FIG. 7). These observations suggest that DIS3L and DIS3L2 regulate the stability of many human miRNAs.

### Example 4: PARN Deadenylates miRNAs to Protect them from PAPD5-Mediated Adenylation and Degradation by DIS3L or DIS3L2

Our analysis of miRNA steady state levels in PARN knockdown and PARN and PAPD5 co-knockdown cells suggests that for a subset of miRNAs, 3′ end adenylation is regulated by the activities of PARN and PAPD5. To assess the effect of PARN and PAPD5 activity on miRNA 3′ ends directly, we sequenced the 3′ end of miR-21-5p and miR-181b-5p in control, PARN knockdown and PARN and PAPD5 co-knockdown cells. For miR-21-5p, we found that in control cells, 5% of the reads represented adenylated miR-21-5p species at the canonical 3′ end (FIG. 2D). In PARN knockdown cells, we observed a two-fold increase in the levels of adenylated ends, demonstrating PARN deadenylates miR-21-5p (FIG. 2D). Finally, PARN and PAPD5 co-knockdown led to a strong decrease in the levels of adenylated reads at the 3′ end of miR-21-5p compared to control cells (FIG. 2D). We observed a similar trend for the 3′ end of miR-181b-5p in various conditions. In PARN knockdown cells, we observed an increase in the proportion of adenylated reads compared to control cells (FIG. 2E). Conversely, in PARN and PAPD5 co-knockdown cells, the percentage of adenylated reads decreased two-fold compared to control cells (FIG. 2E). This provides evidence for PAPD5-mediated oligoadenylation of miRNA 3′ ends and suggests PARN removes oligo (A) tails from the 3′ ends of miRNAs, protecting them from degradation.

Because DIS3L2 knockdown leads to an increase in the steady state levels of several miRNAs (FIG. 2C), we sequenced the 3′ ends of miR-21-5p and miR-181b-5p in DIS3L2 knockdown cells to determine how DIS3L2 affects their 3′ ends. In contrast to previous publications showing that DIS3L2 degrades uridylated let-7 miRNA precursors (Chang, et al., Nature 497:244-48, 2013; Ustianenko, et al., RNA, 19:1632-38, 2013), we found that DIS3L2 knockdown did not lead to an increase in oligo (U) tails at the 3′ end of miR-21-5p; in fact, we were unable to detect any uridylated tails at the 3′ end of miR-21-5p in either control or DIS3L2 knockdown cells. Similarly, the fraction of uridylated miR-181b-5p species remains unchanged between control and DIS3L2 knockdown cells suggesting that DIS3L2's activity on miRNAs is not restricted to uridylated ends.

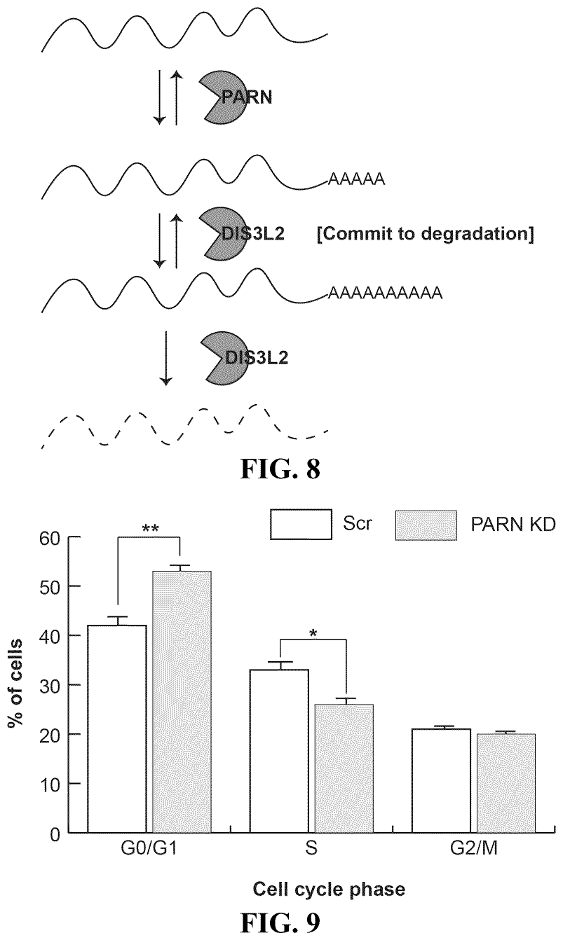

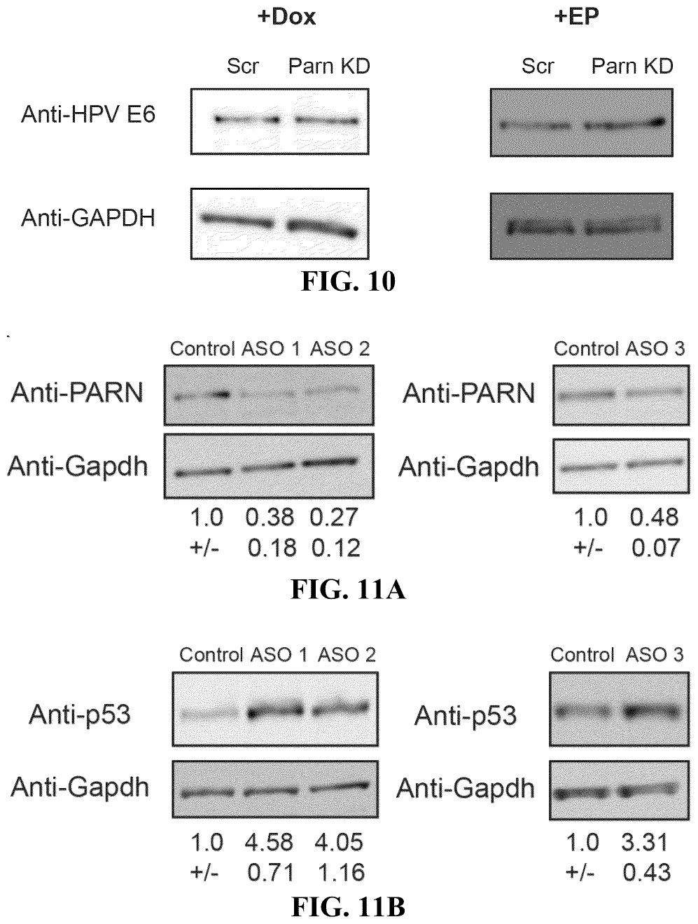

We observed that DIS3L2 knockdown led to a 2X and 2.5X decrease, respectively, in the fraction of oligo (A) tails at the 3′ end of miR-21-5p and miR-181b-5p (FIGS. 2F and 2G). The reduction in oligoadenylation of miRNA 3′ ends upon DIS3L2 knockdown suggests that DIS3L2 actually impedes PARN from removing oligo (A) tails from the 3′ ends of substrate miRNAs. One possibility is that DIS3L2 outcompetes PARN and ‘commits’ the adenylated miRNA species for degradation (FIG. 8). In the absence of DIS3L2, PARN can deadenylate the 3′ ends of miRNAs, leading to a reduction in the fraction of oligoadenylated species for miRNAs in DIS3L2 depleted cells.

miR-181b-5p levels, but not miR-21-5p levels, were affected by the activity of DIS3L. We found that DIS3L knockdown led to a 1.5-fold increase in the fraction of oligo (A) reads at the 3′ end of miR-181b-5p (FIG. 2H). Because DIS3L prefers oligoadenylated substrates for its activity, this observation suggests that DIS3L recognizes oligo (A) tails at the 3′ end of miR-181b-5p and degrades the miRNA through this 3′ to 5′ degradation pathway. These results suggest that PARN, DIS3L, and DIS3L2 affect subsets of miRNAs in human cells.

### Example 5: PARN-Regulated miRNAs Modulate the p53 Signaling Pathway in Human Cells

To determine a biological role of PARN mediated regulation of miRNAs, we examined whether the miRNAs reduced in PARN knockdown cells targeted distinct biological pathways in the cell. KEGG analysis identified several pathways affected by miRNAs altered negatively in PARN knockdown cells, most notably the p53 signaling pathway, as shown in the following table:

| KEGG pathway | p-value | No. of genes |
|---|---|---|
| Fatty acid biosynthesis | 3.60E−16 | 2 |
| Proteoglycans in cancer | 2.95E−06 | 42 |
| Adherens junction | 3.20E−05 | 17 |
| Fatty acid metabolism | 0.001423004 | 6 |
| p53 signaling pathway | 0.002889874 | 20 |

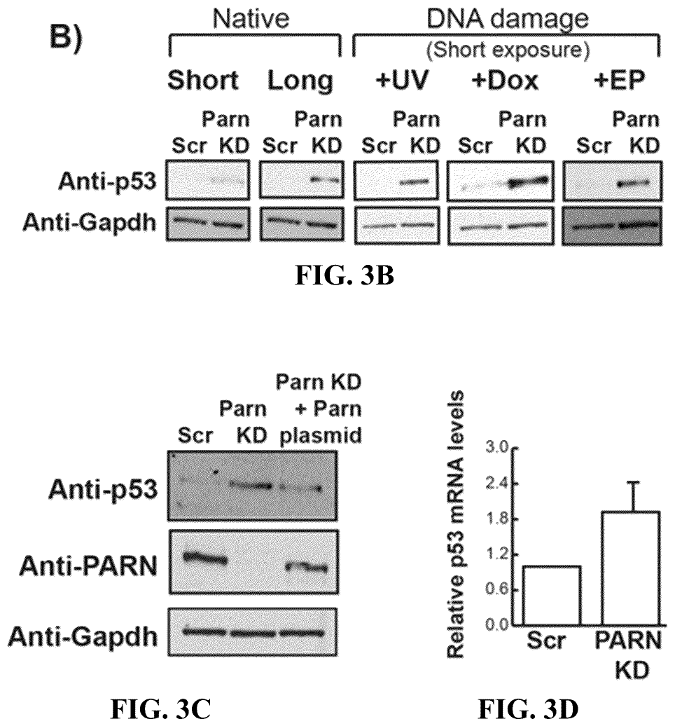

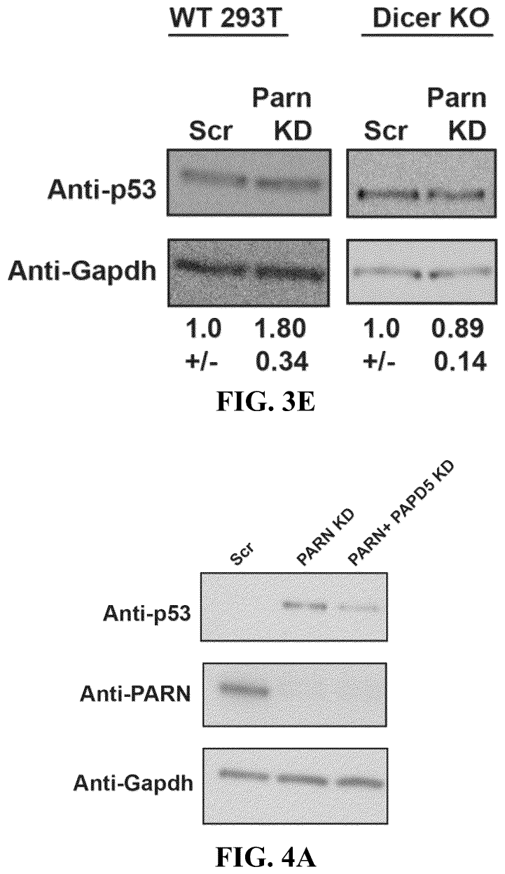

Several miRNAs downregulated upon PARN knockdown, such as miR-380-5p, miR-1285, miR-92, miR-214, miR-485, miR-331, miR-665, miR-3126 and miR-25, have either been shown, or are predicted, to target the TP53 mRNA, which codes for the tumor suppressor protein p53 (FIG. 3A). Similarly, other miRNAs such as miR-660 and miR-32, which repress p53 inhibitors such as MDM2, were upregulated in PARN knockdown cells (FIG. 3A), suggesting PARN-mediated regulation of miRNA levels regulate p53 protein levels in human cells.

Strikingly, we found that p53 levels were upregulated approx. 80-fold in PARN knockdown cells by western blotting (FIG. 3B). Consistent with this observation, PARN knockdown led to an increase in the proportion of cells in the G0/G1 cell cycle phase compared to control cells, which is consistent with p53's role in causing cell cycle arrest in G1 phase (Agarwal et al., 1995; Di Leonardo et al., 1994) (FIG. S5). Moreover, p53 upregulation upon exposure to DNA damage using UV (25 J/m2), Doxorubicin (Dox) or Etoposide (EP) treatment was increased in PARN knockdown cells (approx. 12-fold upon UV, approx. 16-fold upon Dox, and approx. 15-fold upon EP) compared to control (FIG. 3B). Re-introduction of PARN through a plasmid in PARN knockdown cells rescued p53 levels to approx. 70% of PARN knockdown cells, demonstrating that p53 increase is directly dependent on loss of PARN in cells (FIG. 3C).

Our observations suggest that the increase in p53 levels upon PARN knockdown is due to the regulation of miRNA levels by PARN and PAPD5. p53 mRNA levels increased moderately to approx. 1.9 fold in PARN knockdown HeLa cells, (FIG. 3D), and this effect is consistent with the approx. 80-fold increase in p53 levels upon PARN knockdown being accounted for by the loss of translation repression by miRNAs targeting p53 mRNA. Moreover, upregulation of p53 was not caused by a decrease in the HPV E6 oncoprotein which targets p53 for degradation in Hela cells (FIG. S6), suggesting that loss of p53 translation repression in PARN deficient cells leads to the induction of p53 in these cells.

To further verify that the increase in p53 levels upon PARN knockdown is due to disruption of miRNA-mediated p53 translation repression, we examined how PARN knockdown affected p53 protein levels in a Hek293T cell line that lacks miRNAs due to genetic ablation of the Dicer enzyme. Hek293T cells accumulate high amounts of the p53 protein due to the formation of the LTag-p53 complex, which prevents binding of p53 to its target regions on DNA. Despite the high accumulation of p53 protein normally seen in this cell line, we observed that PARN knockdown in wild-type Hek293T cells still led to a 1.8-fold increase in p53 levels. In contrast, p53 levels were unchanged upon PARN knockdown in Dicer knockout cells that lack miRNAs (FIG. 3E). Together, these results suggest PARN depletion leads to an induction of p53 protein and cell cycle arrest in human cells in a miRNA-dependent manner.

### Example 6: PARN and PAPD5 Co-Depletion Rescues p53 Levels in Cancer Cells

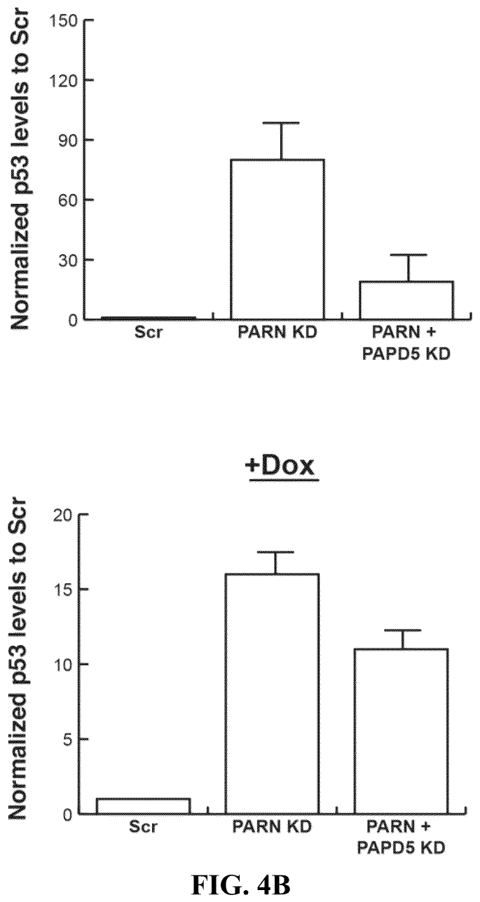

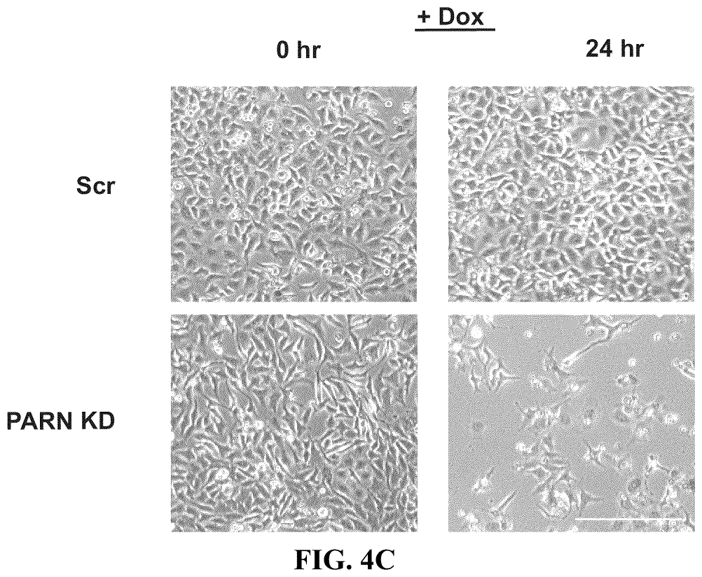

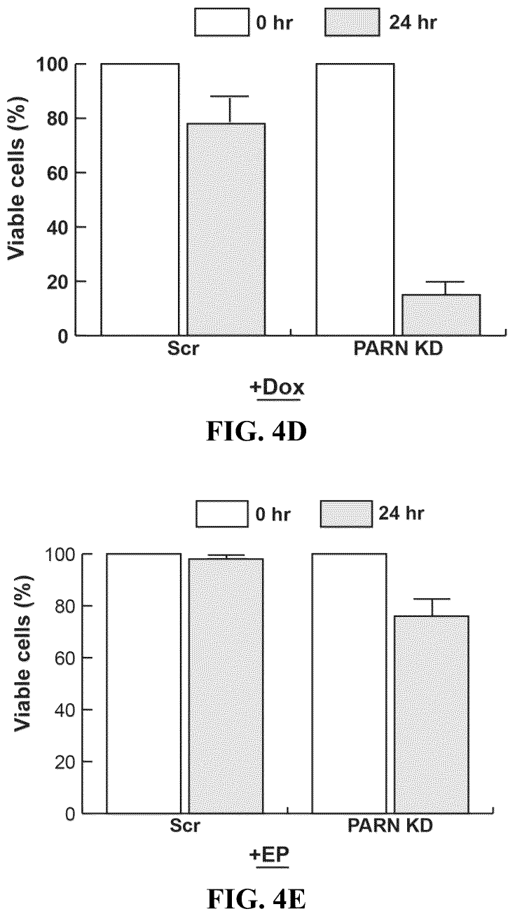

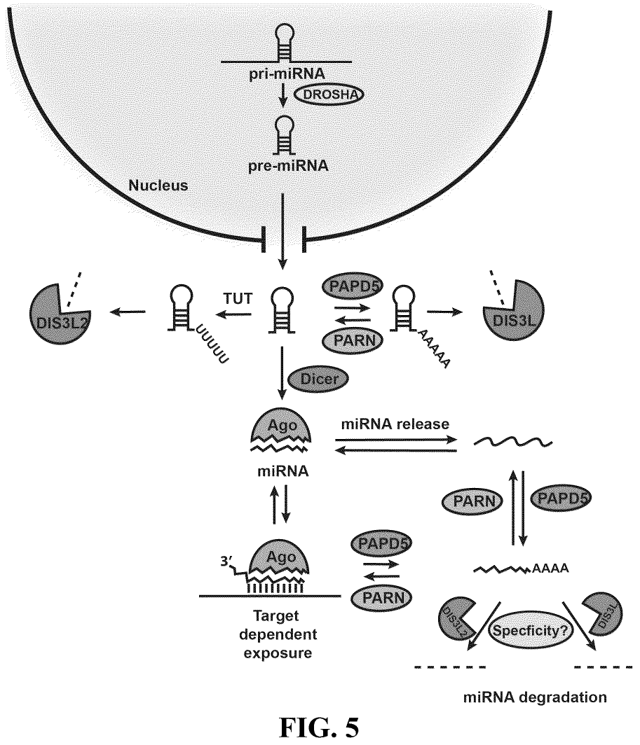

PARN-mediated deadenylation of miRNAs protects them from PAPD5-mediated degradation. Therefore, we asked whether PAPD5 depletion could rescue p53 levels in PARN knockdown cells. We found that PAPD5 co-knockdown, which rescues miRNA levels upon PARN depletion, was also able to rescue p53 levels compared to PARN knockdown with or without DNA damaging agents (FIGS. 4A and 4B). Together, these experiments demonstrate that PARN regulates p53 levels in cells through modulation of miRNAs that repress p53 mRNA translation. Further, this provides a molecular explanation for why DC patient cells containing LOF mutations in PARN also exhibit p53 upregulation and cell cycle. Importantly, the increase in p53 levels may further contribute to the severe phenotype of the disease in these patients.

### Example 7: PARN Knockdown Targets Tumor Protein-53 (p53) mRNA or Protein Levels

A large number of human cancers downregulate the p53 pathway for increased proliferation and resistance to DNA damaging agents. Therefore, upregulation of p53 levels by PARN depletion should make cells more sensitive to commonly used chemotherapeutic agents such as Doxorubicin or Etoposide, which both upregulated p53 levels in PARN knockdown HeLa cells (FIG. 3B). We found that PARN knockdown led to a strong reduction in cell viability upon Doxorubicin treatment compared to control cells (FIGS. 4C and 4D). Similarly, Etoposide treatment also led to a reduction in cell viability upon PARN knockdown compared to control cells (FIG. 4E). These results suggest that selective PARN depletion increases sensitivity of cancer cells to chemotherapeutic drugs such as Doxorubicin and Etoposide, and PARN could be a therapeutic target to reduce the proliferation of tumors that express a reduced level of functional p53 protein.

### Example 8: PARN Inhibition by ASOs in Combination with Doxorubicin Treatment Induces p53 Accumulation in HeLa Cells and Leads to Loss of Viability

Antisense oligonucleotides (ASOs) were designed against the PARN mRNA (NCBI ref: NM_002582) to meet the following criteria:
1. Unique match to the PARN mRNA
2. GC content not more than 50%
3. Avoid more than 4 consecutive G's
4. Target predicted unstructured region in the mRNA

Using these criteria, 7 ASOs were designed to target different regions of the PARN mRNA. Additionally, control ASOs were designed by scrambling the ASO sequence to create non-targeting controls. 50 nM of ASOs were transfected in HeLa cells using DreamFect™ Gold reagent (OZ Biosciences) per the manufacturer's protocols. Cells were harvested after 72 hours to measure PARN or p53 protein levels. When indicated, DNA damage treatment using 1 μM Doxorubicin was performed after 48 hours of ASO transfection.

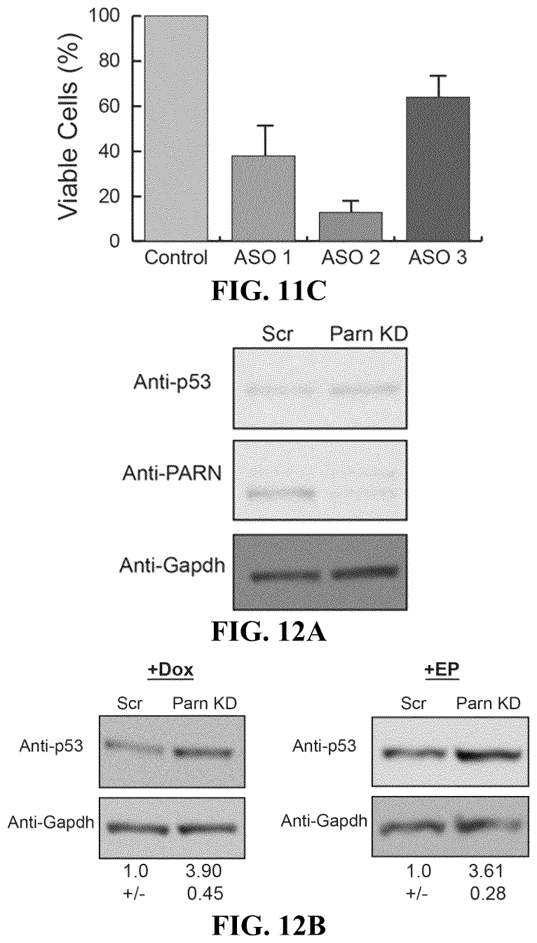

Three ASOs were found to reduce PARN protein levels between 50% to 80% of control ASO transfected cells (FIG. 11A).

(SEQ ID NO. 12) ASO1:
C*T*G*T*C*TTTCTTCTTC*C*T*G*A*T
(SEQ ID NO. 13) ASO2:
C*T*G*T*G*CTGGGAGCTG*T*A*A*A*A
(SEQ ID NO. 14) ASO3:
A*T*T*T*T*TGGAGGGCTG*A*G*A*A*A
(SEQ ID NO. 15) Control:
G*T*C*CTCCCAGTCTTTAA*A*T*T
(‘*’ indicates a phosphorothioate (PS) bond instead of a phosphodiester bond. PS bonds are predicted to increase ASO stability in human cells by reducing exonuclelolytic and endonucleolytic cleavage.)

ASO-mediated PARN knockdown alone led to a modest increase in p53 levels compared to control ASO transfected cells. However, combined with Dox treatment, ASO treatment led to a strong induction of p53 and significant reduction in cell viability (FIGS. 11B and 11C). Therefore, ASO-mediated PARN knockdown leads to p53 induction and loss of cell viability upon Dox treatment which is similar to results obtained with siRNA-mediated PARN knockdown in HeLa cells.

### Example 9: PARN Knockdown in HCT116 Colorectal Carcinoma Cells Induces p53 Accumulation with or without DNA Damage

HCT116 cells were cultured in McCoy's 5A medium supplemented with 10% FBS and Normocin. These cells maintain normal levels of wild-type p53 but have missense mutations in the KRAS oncogene that lead to cancer. For PARN knockdown, approx. 100,000 cells were seeded in a six well plate. After 24 hours, cells were treated with 5 nM of Scr or PARN siRNA using Interferin per the manufacturer's protocols. Cells were harvested three days after transfection. For DNA damage treatment, cells were treated with 5 μM of Dox or 10 μM of EP 48 hours after siRNA transfection and harvested after 24 hours of chemical treatment.

PARN knockdown led to an average 2-fold increase in p53 levels in HCT116 cells, which suggests that PARN regulates p53 levels in cell lines that accumulate normal amount of wild-type p53 (FIG. 12A). Treatment with Dox or EP led to a stronger increase in p53 levels in PARN knockdown cells compared to control cells (average 4-fold increase compared to control) (FIG. 12B). These results suggest that chemotherapeutic agents can be combined with PARN knockdown to induce p53 accumulation in colorectal cancer cells.

### Example 10: PARN Knockdown in U87 Glioblastoma Cells Induces p53 Accumulation and Leads to Loss of Viability when Combined with Doxorubicin Treatment

U87-MG glioblastoma cells expressing wild-type p53 were cultured in Eagle's Modified Essential Medium supplemented with 10% FBS and Normocin. These cells have a homozygous deletion of the PTEN gene, which leads to an aggressive form of glioma (grade IV) and chemoresistance. Approx. 120,000 cells were seeded in a six well plate. After 24 hours, 5 nM of Scr or PARN siRNA was transfected using Interferin per the manufacturer's protocols. Cells were harvested three days after transfection. For DNA damage, cells were treated with 5 μM of Dox or 10 μM of EP 48 hours after siRNA transfection and harvested after 24 hours of chemical treatment. For cell viability analysis, equal number of cells in three biological replicates were stained with Trypan Blue and counted on a hemocytometer.

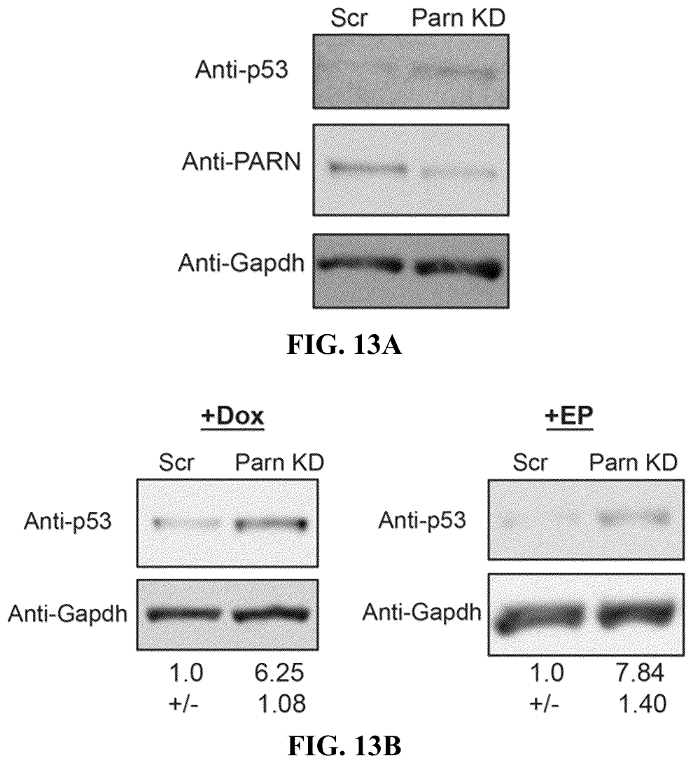

PARN knockdown led to a 3-fold increase in p53 levels along with a visible reduction in cell growth (FIG. 13A). Treatment with Dox led to a approx. 6-fold increase in p53 levels in PARN knockdown cells and a similar result was obtained after EP treatment (approx. 8-fold increase compared to control) (FIG. 13B). These results suggest that PARN inhibition induces p53 accumulation in aggressive forms of glioblastomas.

Glioma cells have been shown to exhibit remarkable resistance to radiotherapy and chemotherapy in previous studies. Because PARN knockdown led to a reduction in cell growth, we measured viability of U87 cells with or without PARN knockdown 24 hours after treatment with Dox. We found that Dox treatment led to a 23% reduction in cell viability for PARN knockdown cells after treatment with Dox, which is supported by an 8-fold increase in p53 levels in these cells (FIG. 13B). This suggests that aggressive gliomas can be targeted by PARN inhibition to reduce cell growth and induce cell death.

The foregoing examples of the present invention have been presented for purposes of illustration and description. Furthermore, these examples are not intended to limit the invention to the form disclosed herein. Consequently, variations and modifications commensurate with the teachings of the description of the invention, and the skill or knowledge of the relevant art, are within the scope of the present invention. The specific embodiments described in the examples provided herein are intended to further explain the best mode known for practicing the invention and to enable others skilled in the art to utilize the invention in such, or other, embodiments and with various modifications required by the particular applications or uses of the present invention. It is intended that the appended claims be construed to include alternative embodiments to the extent permitted by the prior art.

### REFERENCES

ADDIN PAPERS2_CITATIONS <papers2_bibliography/>1. Shukla, S., Schmidt, J. C., Goldfarb, K. C., Cech, T. R. & Parker, R. Inhibition of telomerase RNA decay rescues telomerase deficiency caused by dyskerin or PARN defects. Nature Structural & Molecular Biology 23, 286-292 (2016).
2. Shukla, S. & Parker, R. PARN Modulates Y RNA Stability and Its 3′-End Formation. Molecular and Cellular Biology 37, c00264-17-22 (2017).
3. Nguyen, D. et al. A Polyadenylation-Dependent 3&prime; End Maturation Pathway Is Required for the Synthesis of the Human Telomerase RNA. CellReports 13, 2244-2257 (2015).
4. Moon, D. H. et al. Poly (A)-specific ribonuclease (PARN) mediates 3′-end maturation of the telomerase RNA component. Nature Genetics 47, 1482-1488 (2015).
5. Tseng, C.-K. et al. Human Telomerase RNA Processing and Quality Control. CellReports 13, 2232-2243 (2015).
6. Tang, W., Tu, S., Lee, H.-C., Weng, Z. & Mello, C. C. The RNase PARN-1 Trims piRNA 3&prime; Ends to Promote Transcriptome Surveillance in C.&nbsp; elegans. Cell 164, 974-984 (2016).
7. Izumi, N. et al. Identification and Functional Analysis of the Pre-piRNA 3&prime; Trimmer in Silkworms. Cell 164, 962-973 (2016).
8. Katoh, T., Hojo, H. & Suzuki, T. Destabilization of microRNAs in human cells by 3′ deadenylation mediated by PARN and CUGBP1. Nucleic Acids Research 43, 7521-7534 (2015).
9. Jonas, S. & Izaurralde, E. Towards a molecular understanding of microRNA-mediated gene silencing. Nature Reviews Genetics 16, 421-433 (2015).
10. Ha, M. & Kim, V. N. Regulation of microRNA biogenesis. Nat Rev Mol Cell Biol 15, 509-524 (2014).
11. Boele, J. et al. PAPD5-mediated 3′ adenylation and subsequent degradation of miR-21 is disrupted in proliferative disease. Proceedings of the National Academy of Sciences 111, 11467-11472 (2014).
12. Berndt, H. et al. Maturation of mammalian H/ACA box snoRNAs: PAPD5-dependent adenylation and PARN-dependent trimming. RNA 18, 958-972 (2012).
13. Zinder, J. C. & Lima, C. D. Targeting RNA for processing or destruction by the eukaryotic RNA exosome and its cofactors. Genes & Development 31, 88-100 (2017).
14. Swarbrick, A. et al. miR-380-5p represses p53 to control cellular survival and is associated with poor outcome in MYCN-amplified neuroblastoma. Nat. Med. 16, 1134-1140 (2010).
15. Vlachos, I. S. et al. DIANA-miRPath v3.0: deciphering microRNA function with experimental support. Nucleic Acids Research 43, W460-6 (2015).
16. Liu, J., Zhang, C., Zhao, Y. & Feng, Z. MicroRNA Control of p53. J. Cell. Biochem. 118, 7-14 (2016).
17. Agarwal, V., Bell, G. W., Nam, J.-W. & Bartel, D. P. Predicting effective microRNA target sites in mammalian mRNAs. eLife 4, 101 (2015).
18. Zhang, L.-N. & Yan, Y.-B. Depletion of poly (A)-specific ribonuclease (PARN) inhibits proliferation of human gastric cancer cells by blocking cell cycle progression. BBA-Molecular Cell Research 1853, 522-534 (2015).
19. Bogerd, H. P., Whisnant, A. W., Kennedy, E. M., Flores, O. & Cullen, B. R. Derivation and characterization of Dicer- and microRNA-deficient human cells. RNA 20, 923-937 (2014).
20. Lilyestrom, W., Klein, M. G., Zhang, R., Joachimiak, A. & Chen, X. S. Crystal structure of SV40 large T-antigen bound to p53: interplay between a viral oncoprotein and a cellular tumor suppressor. Genes & Development 20, 2373-2382 (2006).
21. Deppert, W., Steinmayer, T. & Richter, W. Cooperation of SV40 large T antigen and the cellular protein p53 in maintenance of cell transformation. Oncogene 4, 1103-1110 (1989).
22. Tummala, H. et al. Poly (A)-specific ribonuclease deficiency impacts telomere biology and causes dyskeratosis congenita. J. Clin. Invest. 125, 2151-2160 (2015).
23. Dhanraj, S. et al. Bone marrow failure and developmental delay caused by mutations in poly (A)-specific ribonuclease (PARN). J Med Genet 52, 738-748 (2015).
24. Joerger, A. C. & Fersht, A. R. The p53 Pathway: Origins, Inactivation in Cancer, and Emerging Therapeutic Approaches. Annu. Rev. Biochem. 85, 375-404 (2016).
25 Bieging, K. T., Mello, S. S. & Attardi, L. D. Unravelling mechanisms of p53-mediated tumour suppression. Nat. Rev. Cancer 14, 359-370 (2014).
26. Maragozidis, P. et al. Alterations of deadenylase expression in acute leukemias: evidence for poly (a)-specific ribonuclease as a potential biomarker. Acta Haematol. 128, 39-46 (2012).
27. Maragozidis, P. et al. Poly (A)-specific ribonuclease and Nocturnin in squamous cell lung cancer: prognostic value and impact on gene expression. Mol. Cancer 14, 187 (2015).
28. Uhlén, M. et al. Proteomics. Tissue-based map of the human proteome. Science 347, 1260419-1260419 (2015).
29. Uhlen, M. et al. A pathology atlas of the human cancer transcriptome. Science 357, eaan2507 (2017).
30 Burroughs, A. M. et al. A comprehensive survey of 3′ animal miRNA modification events and a possible role for 3′ adenylation in modulating miRNA targeting effectiveness. Genome Research 20, 1398-1410 (2010).
31. Landgraf, P. et al. A Mammalian microRNA Expression Atlas Based on Small RNA Library Sequencing. Cell 129, 1401-1414 (2007).
32. Wyman, S. K. et al. Post-transcriptional generation of miRNA variants by multiple nucleotidyl transferases contributes to miRNA transcriptome complexity. Genome Research 21, 1450-1461 (2011).
33. Zhang, X. et al. PARN deadenylase is involved in miRNA-dependent degradation of TP53 mRNA in mammalian cells. Nucleic Acids Research 43, 10925-10938 (2015).
34. Devany, E., Zhang, X., Park, J. Y., Tian, B. & Kleiman, F. E. Positive and negative feedback loops in the p53 and mRNA 3′ processing pathways. Proc. Natl. Acad. Sci. U.S.A. 110, 3351-3356 (2013).
35. Goldfarb, K. C. & Cech, T. R. 3′ terminal diversity of MRP RNA and other human noncoding RNAs revealed by deep sequencing. BMC Mol. Biol. 14, 23 (2013).

What is claimed is:

1. A method of treating a subject having a cancer, the method comprising:
a) obtaining a test sample from the subject;
b) assaying the test sample to determine the levels of one or more of the following: poly (A)-specific ribonuclease (PARN), and phosphorylated PARN, wherein the level of PARN is performed by Northern blot analysis, Western blot analysis, quantitative or semi-quantitative reverse transcription-polymerase chain reaction (RT-PCR), ligase chain reaction (LCR), transcription-mediated amplification (TMA), strand displacement amplification (SDA), and nucleic acid sequence-based amplification (NASBA);
c) producing an expression profile of the PARN from the test sample;
d) compare the expression profile of c) to a control sample, wherein the control sample; and
e) determining that the cancer is a chemotherapeutically-sensitive cancer where the levels of PARN in the test sample are significantly higher than the control resulting in a loss of function or activity of tumor protein-53 (p53); and
f) treating the subject with a chemotherapeutic cancer therapy selected from: alemtuzumab, altretamine, aminoglutethimide, amsacrine, anastrozole, azacitidine, bleomycin, bicalutamide, busulfan, capecitabine, carboplatin, carmustine, celecoxib, chlorambucil, 2-chlorodeoxyadenosine, cisplatin, colchicine, cyclophosphamide, cytarabine, cytoxan, dacarbazine, dactinomycin, daunorubicin, docetaxel, doxorubicin, epirubicin, estramustine phosphate, etodolac, etoposide, exemestane, floxuridine, fludarabine, 5-fluorouracil, flutamide, formestane, gemcitabine, gentuzumab, goserelin, hexamethylmelamine, hydroxyurea, hypericin, ifosfamide, imatinib, interferon, irinotecan, letrozole, leuporelin, lomustine, mechlorethamine, melphalen, mercaptopurine, 6-mercaptopurine, methotrexate, mitomycin, mitotane, mitoxantrone, nilutamide, nocodazole, paclitaxel, pentostatin, procarbazine, raltitrexed, rituximab, rofecoxib, streptozocin, tamoxifen, temozolomidc, teniposide, 6-thioguanine, topotecan, toremofme, trastuzumab, vinblastine, vincristine, vindesine, and vinorelbine.

2. The method of claim XX100, wherein said step of assaying comprises detecting the amount of mRNA transcripts in the sample encoding a PARN protein according to SEQ ID NO's. 1-4.

3. The method of claim XX100, wherein said wherein said step of assaying comprises detecting the amount of PARN protein in the sample according to SEQ ID NO's. 1-4.

4. The method of claim XX100, wherein said cancer is selected from the group consisting of: acoustic neuroma, acute leukemia, acute lymphocytic leukemia, acute monocytic leukemia, acute myeloblastic leukemia, acute myelocytic leukemia, acute myelomonocytic leukemia, acute promyelocytic leukemia, acute erythroleukemia, adenocarcinoma, angiosarcoma, astrocytoma, basal cell carcinoma, bile duct carcinoma, bladder carcinoma, brain cancer, breast cancer, bronchogenic carcinoma, cervical cancer, chondrosarcoma, chordoma, choriocarcinoma, chronic leukemia, chronic lymphocytic leukemia, chronic myelocytic leukemia, colon cancer, colon carcinoma, craniopharyngioma, cystadenocarcinoma, embryonal carcinoma, enotheliosarcoma, ependymoma, epithelial carcinoma, Ewing's tumor, glioma, heavy chain disease, hemangioblastoma, hepatoma, Hodgkin's disease, large cell carcinoma, leiomyosarcoma, liposarcoma, lung cancer, lung carcinoma, lymphangioendotheliosarcoma, lymphangiosarcoma, macroglobulinemia, medullary carcinoma, medulloblastoma, melanoma, meningioma, mesothelioma, myxosarcoma, neuroblastoma, non-Hodgkin's disease, oligodendroglioma, osteogenic sarcoma, ovarian cancer, pancreatic cancer, papillary adenocarcinomas, papillary carcinoma, pinealoma, polycythemia vera, prostate cancer, rhabdomyosarcoma, renal cell carcinoma, retinoblastoma, schwannoma, sebaceous gland carcinoma, seminoma, small cell lung carcinoma, squamous cell carcinoma, sweat gland carcinoma, synovioma, testicular cancer, uterine cancer, Waldenstrom's fibrosarcoma, and Wilm's tumor.

5. A method of treating a subject having a cancer, the method comprising:
a) obtaining a test sample from the subject;
b) assaying the test sample to determine the levels of one or more of the following: poly (A)-specific ribonuclease (PARN), and phosphorylated poly (A)-specific ribonuclease (PARN), wherein the level of PARN is performed by Northern blot analysis, Western blot analysis, quantitative or semi-quantitative reverse transcription-polymerase chain reaction (RT-PCR), ligase chain reaction (LCR), transcription-mediated amplification (TMA), strand displacement amplification (SDA), and nucleic acid sequence-based amplification (NASBA);
c) producing an expression profile of the PARN from the test sample;
d) compare the expression profile of c) to a control sample;
e) determining that the cancer is a chemotherapeutically-resistant cancer where the levels of PARN in the test sample are significantly lower than the control sample;
f) treating the subject with a non-chemotherapeutic cancer therapy selected from: radiotherapy, surgery, immunotherapy, kinase inhibition, monoclonal antibody therapy, or a combination thereof; ceasing chemotherapeutic treatments on the subject, or inhibiting the activity of expression of PARN.

6. The method of claim XX100, wherein said step of assaying comprises detecting the amount of mRNA transcripts in the sample encoding a PARN protein according to SEQ ID NO's. 1-4, or a functional fragment thereof.

7. The method of claim XX100, wherein said wherein said step of assaying comprises detecting the amount of PARN protein in the sample according to SEQ ID NO's. 1-4, or a functional fragment thereof.

8. The method of claim XX100, wherein said cancer is selected from the group consisting of: acoustic neuroma, acute leukemia, acute lymphocytic leukemia, acute monocytic leukemia, acute myeloblastic leukemia, acute myelocytic leukemia, acute myelomonocytic leukemia, acute promyelocytic leukemia, acute erythroleukemia, adenocarcinoma, angiosarcoma, astrocytoma, basal cell carcinoma, bile duct carcinoma, bladder carcinoma, brain cancer, breast cancer, bronchogenic carcinoma, cervical cancer, chondrosarcoma, chordoma, choriocarcinoma, chronic leukemia, chronic lymphocytic leukemia, chronic myelocytic leukemia, colon cancer, colon carcinoma, craniopharyngioma, cystadenocarcinoma, embryonal carcinoma, endotheliosarcoma, ependymoma, epithelial carcinoma, Ewing's tumor, glioma, heavy chain disease, hemangioblastoma, hepatoma, Hodgkin's disease, large cell carcinoma, leiomyosarcoma, liposarcoma, lung cancer, lung carcinoma, lymphangioendotheliosarcoma, lymphangiosarcoma, macroglobulinemia, medullary carcinoma, medulloblastoma, melanoma, meningioma, mesothelioma, myxosarcoma, neuroblastoma, non-Hodgkin's disease, oligodendroglioma, osteogenic sarcoma, ovarian cancer, pancreatic cancer, papillary adenocarcinomas, papillary carcinoma, pinealoma, polycythemia vera, prostate cancer, rhabdomyosarcoma, renal cell carcinoma, retinoblastoma, schwannoma, sebaceous gland carcinoma, seminoma, small cell lung carcinoma, squamous cell carcinoma, sweat gland carcinoma, synovioma, testicular cancer, uterine cancer, Waldenstrom's fibrosarcoma, and Wilm's tumor.

9. A kit for diagnosing a chemotherapy-resistant or chemotherapy-sensitive cancer in a patient comprising: one or more reagent(s) capable of measuring one or more feature(s) in a cancer cell(s) from said patient selected from the group consisting of: levels of cytoplasmic or nuclear PARN protein; levels of PARN protein or RNA; levels of phosphorylated PARN protein in the cytoplasm or nucleus; and, instructions for using these reagents to determine the presence of a chemotherapy-resistant or chemotherapy-sensitive cancer in said patient.

10. The kit of claim 9, wherein said one or more reagent(s) in (a) are selected from the group consisting of: an antibody that binds phosphorylated, nonphosphorylated, or total PARN protein; an antibody binding to p53 protein; an oligonucleotide comprising a sequence complementary to a nucleic acid sequence encoding a wild type PARN protein; one or more nucleic acid primer(s) complementary to a nucleic acid sequence encoding a wild type PARN protein.

11. The kit of claim 10, wherein said PARN protein comprises a protein according to SEQ ID NO's. 1-4, or a functional fragment thereof, or an oligonucleotide encoding a PARN protein according to SEQ ID NO's. 1-4.


SEQUENCE LISTING (15 sequences)

SEQ ID NO: 1 - protein, 639 aa, Homo sapiens
MEIIRSNFKSNLHKVYQAIEEADFFAIDGEFSGISDGPSVSALTNGFDTPEERYQKLKKHSMDFLLFQFGLCTFKYDYTDSKYITKSFNFYVFPKPFNRSSPDVKFVCQSSSIDFLASQGFDFNKVFRNGIPYLNQEEERQLREQYDEKRSQANGAGALSYVSPNTSKCPVTIPEDQKKFIDQVVEKIEDLLQSEENKNLDLEPCTGFQRKLIYQTLSWKYPKGIHVETLETEKKERYIVISKVDEEERKRREQQKHAKEQEELNDAVGFSRVIHAIANSGKLVIGHNMLLDVMHTVHQFYCPLPADLSEFKEMTTCVFPRLLDTKLMASTQPFKDIINNTSLAELEKRLKETPFNPPKVESAEGFPSYDTASEQLHEAGYDAYITGLCFISMANYLGSFLSPPKIHVSARSKLIEPFFNKLFLMRVMDIPYLNLEGPDLQPKRDHVLHVTFPKEWKTSDLYQLFSAFGNIQISWIDDTSAFVSLSQPEQVKIAVNTSKYAESYRIQTYAEYMGRKQEEKQIKRKWTEDSWKEADSKRLNPQCIPYTLQNHYYRNNSFTAPSTVGKRNLSPSQEEAGLEDGVSGEISDTELEQTDSCAEPLSEGRKKAKKLKRMKKELSPAGSISKNSPATLFEVPDTW

SEQ ID NO: 2 - protein, 639 aa, Homo sapiens; identical to SEQ ID NO: 1

SEQ ID NO: 3 - protein, 639 aa, Homo sapiens; identical to SEQ ID NO: 1

SEQ ID NO: 4 - protein, 639 aa, Homo sapiens; identical to SEQ ID NO: 1

SEQ ID NO: 5 - protein, 393 aa, Homo sapiens [sequence omitted]

SEQ ID NO: 6 - protein, 393 aa, Homo sapiens [sequence omitted]

SEQ ID NO: 7 - protein, 393 aa, Homo sapiens [sequence omitted]

SEQ ID NO: 8 - protein, 393 aa, Homo sapiens [sequence omitted]

SEQ ID NO: 9 - protein, 393 aa, Homo sapiens [sequence omitted]

SEQ ID NO: 10 - protein, 393 aa, Homo sapiens [sequence omitted]

SEQ ID NO: 11 - protein, 393 aa, Homo sapiens [sequence omitted]

SEQ ID NO: 12 - DNA, 20 bp, synthetic construct
  misc_feature 1..20: note = ASO1
  source 1..20: mol_type = other DNA
ctgtctttcttcttcctgat

SEQ ID NO: 13 - DNA, 20 bp, synthetic construct
  misc_feature 1..20: note = ASO2
  source 1..20: mol_type = other DNA
ctgtgctgggagctgtaaaa

SEQ ID NO: 14 - DNA, 20 bp, synthetic construct
  misc_feature 1..20: note = ASO3
  source 1..20: mol_type = other DNA
atttttggagggctgagaaa

SEQ ID NO: 15 - DNA, 20 bp, synthetic construct
  misc_feature 1..20: note = ASO control
  source 1..20: mol_type = other DNA
gtcctcccagtctttaaatt